(19) United States
(12) Patent Application Publication
(10) Pub. No.: US 2023/0149162 A1
(43) Pub. Date: May 18, 2023

(54) PROSTHETIC HEART VALVE

(71) Applicant: Edwards Lifesciences Corporation, Irvine, CA (US)

(72) Inventors: Nikolai Gurovich, Hadera (IL); Tamir S. Levi, Zikhron Yaakov (IL)

(21) Appl. No.: 18/157,044

(22) Filed: Jan. 19, 2023

Related U.S. Application Data
(63) Continuation of application No. PCT/US2021/043112, filed on Jul. 26, 2021.
(60) Provisional application No. 63/056,868, filed on Jul. 27, 2020.

(51) Int. Cl. A61F 2/24

(52) U.S. Cl. CPC A61F 2/2418; A61F 2230/0069

(57) ABSTRACT

An implantable prosthetic device can include a frame movable between a radially compressed configuration and a radially expanded configuration, and a valvular structure. The frame can have an inflow orifice, an outflow orifice, and one or more commissure windows. The valvular structure can include a plurality of leaflets, each leaflet having a main body with an inflow edge, an outflow edge, and a pair of opposing tabs. Each tab can be paired with an adjacent tab of an adjacent leaflet to form a commissure tab assembly, and each commissure tab assembly being coupled to a respective commissure window. Each tab can extend from the main body at an angle such that a radially outer edge of the tab corresponds to a draft angle of the frame.

### CROSS-REFERENCE TO RELATED APPLICATION

This application is a continuation of PCT patent application no. PCT/US2021/043112, filed on Jul. 26, 2021, entitled PROSTHETIC HEART VALVE, which application claims the benefit of U.S. Provisional Application No. 63/056,868, filed on Jul. 27, 2020, entitled SMALL DIAMETER PROSTHETIC VALVE, each of which application is incorporated herein in its entirety by this specific reference.

### FIELD

The present disclosure relates to prosthetic heart valves, and to methods and assemblies for forming leaflet assemblies and attaching the leaflet assemblies to the frame of such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Most expandable, transcatheter heart valves are used for mid to high expansion diameters, for example diameters ranging from 23 to 29 mm. While smaller prosthetic valves available, such as those with diameters of about 20 mm or less, smaller diameter valves are rarely used due to a variety of challenges. For example, smaller diameter prosthetic valves generally cause higher pressure gradients along the prosthetic valve, which can lead to various clinical risks, such as cavitation. Also, smaller prosthetic valves typically have shorter paravalvular sealing elements, which makes it more challenging for the clinician to align the prosthetic valve at the native annulus. Smaller prosthetic valves also can have relatively shorter frames, which can result in leaflet overhang, in which the native valve leaflets overhang the outflow end of the prosthetic valve, thereby disturbing blood flow and/or inhibiting full opening of the prosthetic leaflets. Further, smaller prosthetic valves have relatively smaller frame openings, which can inhibit coronary access through the frame with a catheter in a subsequent procedure. Finally, valve-in-valve procedures involving implantation of a second prosthetic valve in a previously implanted prosthetic valve is more challenging with relatively smaller prosthetic valves because it is more difficult to properly align and orient the second prosthetic valve within the previously implanted prosthetic valve while maintaining access to the coronary ostia.

Accordingly, a need exists for improved prosthetic heart valve leaflet assemblies and methods for assembling the leaflet assemblies to a frame of the prosthetic heart valve.

### SUMMARY

In a representative embodiment, an implantable prosthetic device can comprise a frame movable between a radially compressed configuration and a radially expanded configuration, the frame having an inflow orifice, an outflow orifice, and comprising one or more commissure windows, and a valvular structure comprising a plurality of leaflets. Each leaflet comprising a main body having an inflow edge, an outflow edge, and a pair of opposing tabs extending from opposite sides of the main body, each tab being paired with an adjacent tab of an adjacent leaflet to form a commissure tab assembly, each commissure tab assembly being coupled to a respective commissure window. Wherein each tab extends from the main body at an angle such that a radially outer edge of the tab corresponds to a draft angle of the frame.

In another representative embodiment, an implantable prosthetic device can comprise a cylindrical frame movable between a radially compressed configuration and a radially expanded configuration, and a valvular structure comprising a plurality of leaflets, each leaflet comprising a main body having an inflow edge, an outflow edge, and a pair of opposing tabs extending from opposite sides of the main body. Each tab can extend from the main body such that an outflow edge of the tab is disposed at a 90-degree angle relative to a longitudinal axis of the leaflet.

In another representative embodiment, an implantable prosthetic device can comprise a cylindrical frame movable between a radially compressed configuration and a radially expanded configuration, the frame comprising an inflow orifice and an outflow orifice and a valvular structure comprising a plurality of leaflets. Each leaflet can comprise a main body having an inflow edge and an outflow edge, a pair of opposing lower tabs extending from opposite sides of the main body, and a pair of opposing upper tabs extending from and coupled to the outflow edge of the leaflet via respective neck portions. Each lower tab can extend from the main body such that an outflow edge of the leaflet tab is disposed at a 90-degree angle relative to a longitudinal axis of the leaflet, and each lower tab can be paired with an adjacent upper tab of an adjacent leaflet to form a plurality of commissures, and each upper tab can be folded toward the inflow orifice of the frame such that the neck portion forms a rigid portion extending radially inwardly toward a longitudinal axis of the frame such that the outflow edge of the leaflet defines a selected geometric orifice area (GOA) within the outflow orifice.

In still another representative embodiment, an implantable prosthetic device can comprise a non-cylindrical frame having an inflow orifice and an outflow orifice, the frame movable between a radially compressed configuration and a radially expanded configuration, the frame having a shape in the radially expanded configuration that tapers from a first diameter at the outflow orifice to a second diameter at the inflow orifice, the second diameter being larger than the first diameter, and a valvular structure comprising a plurality of leaflets. Each leaflet can comprise a main body having an inflow edge, an outflow edge, and a pair of opposing tabs extending from opposite sides of the main body, and each tab extends from the main body at an angle such that a radially outer edge of the tab corresponds to a draft angle of the frame.

In a representative embodiment, an implantable prosthetic device can include an annular frame that is movable between a radially compressed configuration and a radially expanded configuration, the frame comprising first, second, and third circumferentially-extending rows of cells, the first row of cells disposed adjacent an outflow end of the frame and a valvular structure comprising a plurality of leaflets, each leaflet having a main body including an inflow edge and an outflow edge, and a pair of opposing tabs extending from opposite sides of the main body, each tab being paired with an adjacent tab of an adjacent leaflet and secured to the frame to form a commissure assembly. The valvular structure is secured to the frame such that a gap is defined between the outflow edges of the leaflets and the outflow end of the frame, and each cell of the first row of cells is configured to be at least twice as wide as a selected coronary catheter.

A representative method can include inserting a distal end of a delivery apparatus into the vasculature of a patient, the delivery apparatus releasably coupled to a guest prosthetic valve movable between a radially compressed and a radially expanded configuration, the prosthetic valve including a frame comprising first, second, and third circumferentially-extending rows of cells, the first row of cells disposed adjacent an outflow end of the frame and configured to be at least twice as wide as a selected coronary catheter, and a valvular structure disposed within the frame and coupled to the frame such that a gap is defined between the outflow edges of the valvular structure and an outflow end of the frame. The method can further include advancing the guest prosthetic valve to a selected implantation site comprising a previously implanted host prosthetic valve, the host prosthetic valve comprising a host frame and a host valvular structure disposed within the host frame, positioning the guest prosthetic valve within the host prosthetic valve, and radially expanding the guest prosthetic valve within the previously implanted host prosthetic valve.

In some embodiments, the host frame comprises first, second, and third circumferentially-extending rows of cells, the first row of cells disposed adjacent an outflow end of the host frame and configured to be at least twice as wide as a selected coronary catheter, and the host valvular structure is coupled to the host frame such that a gap is defined between the outflow edges of the host valvular structure and an outflow end of the host frame. In such embodiments, the method can further comprise inserting the selected coronary catheter through the gap of the guest prosthetic valve and the gap of the host prosthetic valve.

A representative method of assembling a prosthetic heart valve can comprise forming a valvular structure from a plurality of leaflets, each leaflet comprising an inflow edge, an outflow edge, and two opposing tabs, wherein the valvular structure is formed by coupling adjacent tabs of adjacent leaflets to one another to form respective commissures, positioning the valvular structure within a radially expandable and compressible frame, the frame comprising first, second, and third circumferentially-extending rows of cells, the first row of cells disposed adjacent an outflow end of the frame and configured to be at least twice as wide as a selected coronary catheter, and coupling the valvular structure to the frame such that a gap is defined between the outflow edges of each leaflet and an outflow edge of the frame when the valvular structure is in an open configuration.

In another representative embodiment, an assembly can comprise a first implantable prosthetic device and a second implantable prosthetic device. Each implantable prosthetic device can comprise an annular frame that is movable between a radially compressed configuration and a radially expanded configuration, the frame comprising first, second, and third circumferentially-extending rows of cells, the first row of cells disposed adjacent an outflow end of the frame, and a valvular structure comprising a plurality of leaflets. Each leaflet can have a main body including an inflow edge and an outflow edge, and a pair of opposing tabs extending from opposite sides of the main body, each tab being paired with an adjacent tab of an adjacent leaflet and secured to the frame to form a commissure assembly, the valvular structure can be secured to the frame such that a gap is defined between the outflow edges of the leaflets and the outflow end of the frame. Each cell of the first row of cells can be configured to be at least twice as wide as a selected coronary catheter, and the first implantable prosthetic device can be disposed withing the annular frame of the second implantable prosthetic device.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of a prosthetic heart valve, according to one embodiment.

FIG. 2 is a perspective view of the prosthetic heart valve of FIG. 1, shown with the outer skirt removed and one of the leaflets transparent for purposes of illustration.

FIG. 3A is a perspective view of the frame of the prosthetic heart valve of FIG. 1.

FIG. 3B is a side elevation view of a portion of the frame of the prosthetic heart valve of FIG. 1.

FIG. 4 is a perspective view of a valve-in-valve configuration including the prosthetic heart valve of FIG. 1 as the guest valve, according to one embodiment.

FIG. 5 is a perspective view of a valve-in-valve configuration, according to another embodiment.

FIG. 6 is a perspective view of an extreme valve-in-valve configuration, according to still another embodiment.

FIG. 7 is a side elevation view of a prosthetic heart valve, according to one embodiment.

FIG. 8 is a side elevation view of the frame of the prosthetic heart valve of FIG. 7.

FIG. 9 is a side elevation view of a prosthetic heart valve, according to another embodiment.

FIG. 10 is a side elevation view of a portion of the frame of the prosthetic heart valve of FIG. 9.

FIG. 11 is a perspective view of an embodiment of a prosthetic heart valve, according to one embodiment.

FIG. 12 is a side elevational view of a leaflet of the prosthetic heart valve of FIG. 11.

FIG. 13 is a top plan view of the prosthetic heart valve of FIG. 11, with the valvular structure shown in the open configuration.

FIG. 14 is a perspective view of a commissure portion of the prosthetic heart valve of FIG. 11.

FIG. 15 is a perspective view of a prosthetic heart valve, according to another embodiment.

FIG. 16 is a side elevational view of a leaflet of the prosthetic heart valve of FIG. 15.

FIG. 17 is a cross-sectional view of a commissure portion of the prosthetic heart valve of FIG. 15.

FIG. 18 is a top plan view of the prosthetic heart valve of FIG. 18, with the valvular structure shown in the open configuration.

FIG. 19 is a top plan view of a prosthetic heart valve with the valvular structure shown in the open configuration, according to another embodiment.

FIG. 20 is a side elevational view of a leaflet of the prosthetic heart valve of FIG. 19.

FIG. 21 is a side view of an embodiment of a prosthetic valve being implanted within a native aortic valve of a heart, which is partially shown.

FIG. 22 is a side view of an embodiment of a frame of a prosthetic valve implanted within a native aortic valve of a heart, which is partially shown.

FIG. 23 is a side view of an embodiment of an exemplary valve-in-valve configuration implanted within a native aortic valve of a heart, which is partially shown.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In the context of the present application, the terms “lower” and “upper” are used interchangeably with the terms “inflow” and “outflow”, respectively. Thus, for example, the lower end of the valve is its inflow end and the upper end of the valve is its outflow end.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device toward the user, while distal motion of the device is motion of the device away from the user. The terms “longitudinal” and “axial” refer to an axis extending in the proximal and distal directions, unless otherwise expressly defined.

### Examples of the Disclosed Technology

Described herein are examples of prosthetic implants, such as prosthetic valves, that can be implanted within any of the native valves of the heart (e.g., the aortic, mitral, tricuspid and pulmonary valves). The present disclosure also provides frames for use with such prosthetic implants. The frames can comprise struts having different shapes and/or sizes to avoid coronary blockage and native leaflet overhang. The prosthetic heart valves may also include a plurality of leaflets attached to the frame.

The present disclosure also may include leaflet assemblies for prosthetic heart valves, leaflet commissure tab assemblies of a leaflet assembly, and methods for assembling leaflet commissure tab assemblies. The leaflet commissure tab assemblies may include a plurality of leaflet commissure support members. Each leaflet commissure tab assembly can include a pair of adjacent leaflet tabs coupled to one another by the commissure support member. Each leaflet commissure assembly can be formed by folding and securing a tab of each of the leaflets around a corresponding commissure support member. The adjacently arranged valve leaflets can then be coupled to one another, prior to being attached to the frame of the prosthetic heart valve. As a result, a leaflet assembly for a prosthetic heart valve may be more easily assembled off the frame of the prosthetic heart valve and the time and effort for securing the leaflet assembly to the frame of the prosthetic heart valve may be reduced.

Also disclosed herein are various small diameter prosthetic valves (e.g., 20 mm) that can address one or more of the drawbacks associated with known small diameter prosthetic valves. In particular, disclosed embodiments can be configured to reduce pressure gradients, avoid native leaflet overhang, and/or maintain access and blood flow to the coronary arteries, all issues commonly associated with smaller diameter valves. Disclosed embodiments can comprise a plurality of commissure tab assemblies of the leaflet assembly being coupled to the outside surface of the frame. The disclosed commissure tab assemblies can, for example, allow the valve leaflets to open wider than generally allowed in conventional valves, which increases the overall blood flow through the prosthetic valve to reduce high-pressure gradients.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the valves disclosed herein may be used with a variety of implant delivery apparatuses. Though the prosthetic valves shown herein are described as plastically-deformable or balloon-expandable prosthetic valves, it should be noted that the frame shapes and leaflet configurations disclosed herein can be used with any type of prosthetic valve. For example, the frame shapes and leaflet configurations disclosed herein can be used with mechanically-expandable prosthetic heart valves in which the frame is radially expandable via one or more mechanical actuators (such as the prosthetic valves described in U.S. Pat. No. 10,603,165 and U.S. Provisional Application No. 63/085,947, filed Sep. 30, 2020, each of which is incorporated herein by reference in its entirety). The frames of some mechanical valves can comprise pivotable junctions between the struts of the frame, while others can comprise a unitary lattice frame expandable and/or compressible via mechanical means. The frame shapes and leaflet configurations described herein can additionally be used with other types of transcatheter prosthetic valves, including self-expandable prosthetic heart valves in which the frame is made from a shape memory material (e.g., Nitinol), such as disclosed in U.S. Pat. No. 10,098,734, which is incorporated herein by reference in its entirety.

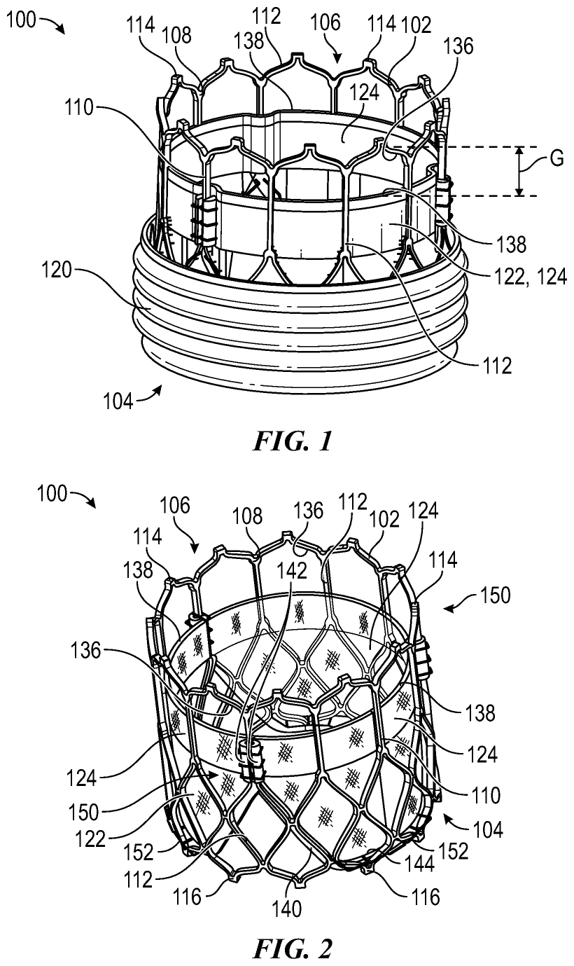

FIGS. 1-2 illustrate an exemplary embodiment of a prosthetic heart valve 100. The prosthetic heart valve 100 can be radially compressible and expandable between a radially compressed configuration and a radially expanded configuration. In particular embodiments, the prosthetic heart valve 100 can be implanted within the native aortic annulus, although it also can be implanted at other locations in the heart, including within the native mitral valve, the native pulmonary valve, and the native tricuspid valve. The prosthetic heart valve 100 can comprise an annular stent or frame 102 having a first or inflow end 104, a second or outflow end 106, a radially inner surface 108, and a radially outer surface 110. A valvular structure 122 comprising a plurality of leaflets 124, can be disposed within the frame 102, as described in more detail below. The valvular structure 122 can be configured to regulate the flow of blood through the prosthetic valve 100 from the inflow end 104 to the outflow end 106. For purposes of illustration, the rearmost leaflet in FIG. 2 is shown transparently.

The outflow end 106 can be coupled to a delivery apparatus for delivering and implanting the prosthetic heart valve within the native aortic valve is a transfemoral, retrograde delivery approach. Thus, in the delivery configuration of the prosthetic heart valve, the outflow end 106 is the proximal-most end of the prosthetic valve. In other embodiments, the inflow end 104 can be coupled to the delivery apparatus, depending on the particular native valve being replaced and the delivery technique that is used (e.g., trans-septal, transapical, etc.). For example, the inflow end 104 can be coupled to the delivery apparatus (and therefore is the proximal-most end of the prosthetic heart valve in the delivery configuration) when delivering the prosthetic heart valve to the native mitral valve via a trans-septal delivery approach.

As shown in FIGS. 1 and 2, the frame 102 can include a plurality of interconnected lattice struts 112 arranged in a lattice-type pattern and forming a plurality of apices 114 at the outflow end 106 of the prosthetic valve 100. The struts 112 can also form similar apices 116 (FIG. 2) at the inflow end 104 of the prosthetic valve 100. The frame 102 can be made of any of various suitable plastically expandable materials, such as stainless steel or a cobalt chromium alloy, and/or self-expanding materials, such as a nickel titanium alloy (“NiTi”), for example Nitinol. When constructed of a plastically expandable material, the frame 102 (and thus the prosthetic valve 100) can be crimped to a radially compressed state on a delivery catheter and then expanded inside a patient by an inflatable balloon or any suitable expansion mechanism, such as the mechanical expansion mechanisms described in U.S. Provisional Application No. 63/085,947, filed Sep. 30, 2020 or U.S. Provisional Application No. 63/179,766 filed Apr. 26, 2021, which is incorporated herein by reference in its entirety. When constructed of a self-expandable material, the frame 102 (and thus the prosthetic valve 100) can be crimped to a radially compressed state and restrained in the compressed state by insertion into a sheath or equivalent mechanism of a delivery catheter. Once inside the body, the prosthetic valve 100 can be advanced from the delivery sheath, which allows the valve to expand to its functional size.

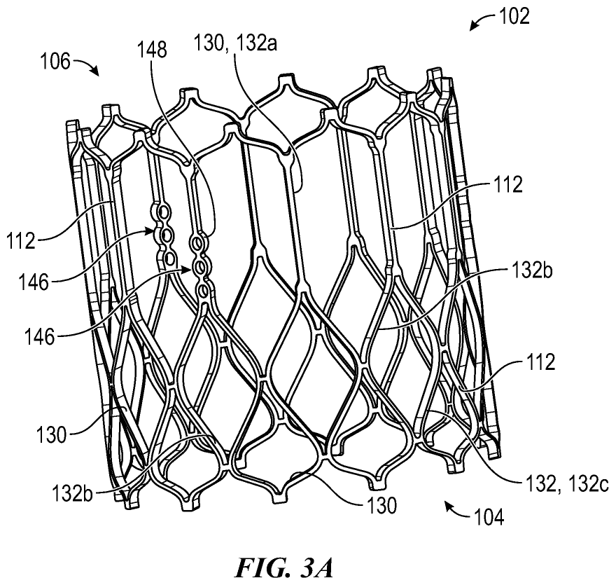

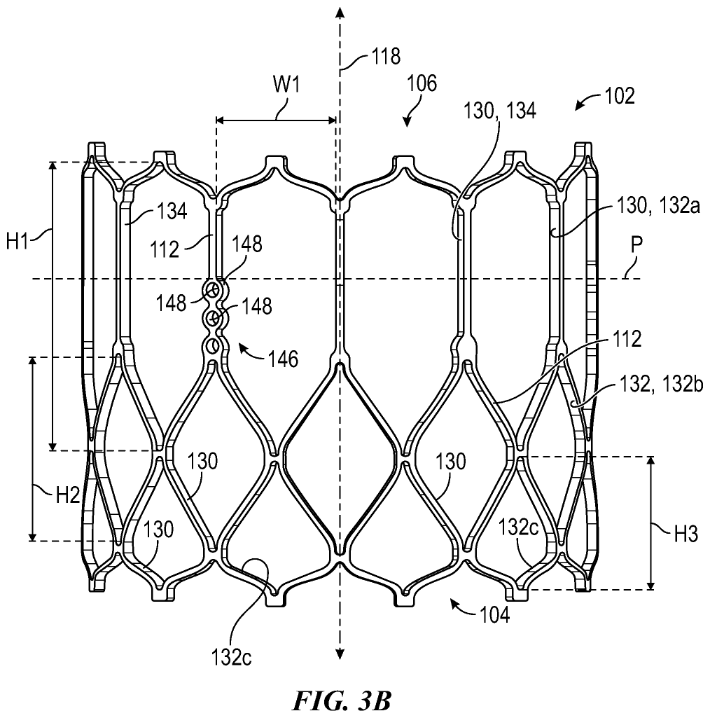

In the illustrated embodiment, the struts 112 are pivotable or bendable relative to each other to permit radial expansion and contraction of the frame 102. For example, the frame 102 can be formed (e.g., via laser cutting, electroforming or physical vapor deposition) from a single piece of material (e.g., a metal tube). As such, the inflow end 104 and the outflow end 106 of the frame 102 can move axially parallel to the longitudinal axis 118 (FIG. 3B) of the prosthetic valve 100 as it is radially expanded or compressed, such as during assembly, preparation, or implantation of the prosthetic valve 100.

In other embodiments, the frame 102 can be constructed by forming individual components (e.g., the struts and fasteners of the frame) and then mechanically assembling and connecting the individual components together. For example, the struts 112 can be pivotably coupled to one another at one or more pivot joints or pivot junctions along the length of each strut. Each of the pivot joints or pivot junctions (e.g., hinges) can allow the struts 112 to pivot relative to one another as the frame 102 is radially expanded or compressed. Further details regarding the construction of the frame and the prosthetic valve are described in U.S. Patent Publication No. 2018/0028310, which is incorporated herein by reference in its entirety. Other frames that can be implemented in the prosthetic valve are disclosed in U.S. Pat. Nos. 9,393,110 and 9,155,619, and 10,603,165, which are incorporated herein by reference in their entireties.

As shown in FIG. 1, the prosthetic valve 100 can also include an outer skirt 120 mounted on the outer surface 110 of the frame 102. The outer skirt 120 can function as a sealing member for the prosthetic valve 100 by sealing against the tissue of the native valve annulus and helping reduce paravalvular leakage past the prosthetic valve. The outer skirt 120 can be formed from any of various suitable biocompatible materials, including any of various synthetic materials (e.g., PET) or natural tissue (e.g., pericardial tissue). The outer skirt 120 can be mounted to the frame 102 using sutures, an adhesive, welding, and/or other means for attaching the outer skirt 120 to the frame 102.

Selection of the height of the frame of a prosthetic valve is an important consideration, especially for smaller diameter prosthetic valves (e.g., 20 mm or smaller). Generally speaking, the frame of a prosthetic valve desirably should be short enough to avoid extending beyond the sinotubular junction (STJ) line and avoid tilting of the prosthetic valve from its intended implanted orientation, yet long enough to avoid native leaflet overhang. It has been found that for patients needing a relatively smaller prosthetic valve (20 mm or smaller), a prosthetic valve having a height of about 14 mm or shorter can increase the risk of leaflet overhang while a prosthetic valve having a height of over 18 mm is likely to extend beyond the STJ line.

FIGS. 3A-3B show the frame 102 with the valvular structure 122 and skirt 120 removed for purposes of illustration. As best seen in FIG. 3A, the struts 112 form a plurality of closed cells 130 arranged in a plurality of circumferentially-extending rows 132 of cells. Each row 132 of cells 130 can get progressively larger from the inflow end 104 to the outflow end 106. In the illustrated embodiment, the struts 112 define three rows of cells, including a first row 132a adjacent the outflow end 106 of the frame, a second row 132b, and a third row 132c adjacent the inflow end 104 of the frame 102. In other embodiments, the frame 102 can have a greater or fewer number of rows 132.

FIG. 3B illustrates a partial view of the frame 102. While only one side of the frame 102 is depicted in FIG. 3B, it should be appreciated that frame 102 forms an annular structure as shown in FIG. 3A having an opposite side that is identical (or substantially identical) to the portion shown. As shown in FIG. 3B, the cells 130 in the row 132a adjacent the outflow end 106 of the frame 102 can have a relatively larger open cell area compared to the cells of rows 132b and 132c. Accordingly, the cells 130 in row 132a can be referred to as “larger” or “elongated” cells 134. In the illustrated embodiment, the elongated cells 134 have a height H₁ greater than a height H₂ of the cells 130 of row 132b, and/or a height H₃ of the cells 130 of row 132c. The elongated cells 134 can have a width W₁ that is at least twice the width of a coronary catheter (e.g., a 6 Fr coronary catheter). The height of the elongated cells 134, in combination with the positioning of the valvular structure 122 within the frame 102 defines a gap G (FIG. 1) between the outflow end 136 of the elongated cells 134 and the outflow edge 138 of the leaflets 124 configured to accommodate a coronary catheter there-through, as described further below.

The smaller cells, such as the cells in rows 132b, 132c in the illustrated embodiment, can have a relatively stronger structural strength than larger cells 134. Accordingly, the frame 102 can be positioned within the native annulus such that the smaller cells 130 in rows 132b, 132c bear a greater amount of the radial force applied by the native annulus than the larger cells.

As mentioned previously and shown in FIGS. 1-2, the prosthetic valve 100 can also include a valvular structure 122 (shown in the open configuration) which is coupled to and supported by the frame 102. The valvular structure 122 can include, for example, a leaflet assembly comprising one or more leaflets 124 made of a flexible material. The leaflets 124 can be made in whole or in part, from biological material, bio-compatible synthetic materials, or other such materials. Suitable biological material can include, for example, bovine pericardium (or pericardium from other sources).

The leaflets 124 can be secured to one another at their adjacent sides to form commissures 126, each which can be secured to a respective commissure post 128. Selection of the height of the individual leaflets is an important consideration for smaller diameter prosthetic valves. Generally speaking, the leaflets should be high enough to promote full closure of the leaflets during diastole, for example, to prevent unwanted back flow through the prosthetic valve. On the other hand, the leaflets should also be low enough as to not block access to the coronary arteries when in an opened and closed configuration.

Referring to FIG. 2, each leaflet 124 can have a curved, scallop shape including a lower cusp portion 140 extending between first and second tabs 142 of the leaflet 124 and an outflow edge 138 (also referred to as a coaptation edge) that contacts respective outflow edges of the other leaflets during diastole. The lower cusp portion 140 can comprise an inflow edge 144 offset from the outflow edge 138 along a longitudinal axis of the valve 100. The inflow edge 144 can be aligned with (or substantially aligned with) and coupled to the inflow end 104 of the frame 102, and the outflow edge 138 can be disposed such that it is located between the inflow end 104 and the outflow end 106 of the frame 102, defining a gap G between the outflow edge 138 of the leaflet 124 and the outflow end 106 of the frame when the valvular structure 122 is in the open configuration. The gap G can remain open and accessible during the working cycle of the prosthetic valve 100, thereby reducing potential blockage of the coronary arteries.

The valvular structure 122 can be coupled to the frame 102 via one or more commissure posts 146. As shown in FIG. 3B, selected struts 112 of the elongated cells 134 can be configured as commissure posts 146. Each commissure post 146 can comprise a plurality of apertures 148. The commissure post 146 can be disposed such that it is spaced apart from the outflow end 106 of the frame 102 along a longitudinal axis 118 of the valve 100. In the illustrated embodiment, an outflow edge 148 of the commissure post 146 can be disposed such that it substantially aligns with a plane P that is perpendicular to the longitudinal axis of the frame and bisects each of the elongated openings 134. However, in other embodiments, the commissure post 146 can be disposed at any location along the height of the elongated cell 134.

In the illustrated embodiment, the frame 102 can comprise three commissure posts 146 spaced apart from one another about the circumference of the frame 102. However, in other embodiments, the frame 102 can comprise a greater or fewer number of commissure posts and the commissure posts 146 can be disposed at any position about the circumference of the frame 102. In the illustrated embodiment, each commissure post 146 comprises three apertures 148 extending along a height of the commissure post 146. One or more leaflets 124 of the valvular structure 122 can be sutured to the frame 102 via the plurality of apertures 148, as shown in FIGS. 1-2.

Coupling the leaflets 124 to the apertures 148 of the commissure post 146 advantageously does not require the use of an intermediate cloth layer. Rather, each tab 142 of a leaflet 124 can be coupled to a tab 142 of an adjacent leaflet 124 to form a commissure 150. Each commissure 150 can be sutured directly to the frame 102 at a respective commissure post 146. Likewise, the cusp edge 140 of each leaflet 124 can be sutured along the scallop line directly to the frame 102. Eliminating intermediate cloth portions by suturing the leaflets 124 directly to the frame 102 can advantageously prevent or mitigate tissue ingrowth along the cusp edge portion 140 and/or the commissures 150. Such a configuration is feasible in small-diameter prosthetic valves, such as prosthetic valve 100, due to the relatively lower stresses experienced by the leaflets 124 in such prosthetic valves, the lower stresses resulting from systolic and/or diastolic pressure being applied over a relatively smaller area, when compared to a non-small diameter prosthetic valve.

In some embodiments, the valvular structure 122 can be coupled to the frame 102 using, for example, a wide, thick suture 152 (FIG. 2). Such a suture 152 can advantageously prevent or mitigate tearing of the leaflets 124 along the portions of the leaflets coupled to the frame.

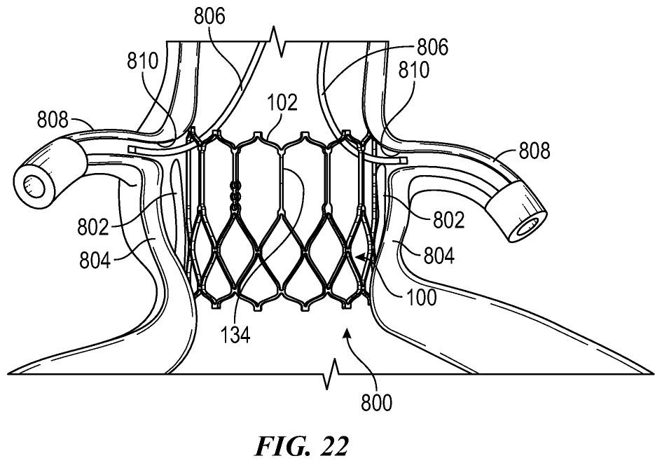

The height H₁ of the elongated cells 134 in combination with the position of the commissure posts 146 and thereby the outflow edges 138 of the leaflets 124 allows access to the coronary vessel when the prosthetic valve 100 is implanted within the native annulus of a patient. For example, in some instances a patient may require implantation of a coronary stent (or other procedure that requires access to the coronary vessel) after a prosthetic heart valve, such as prosthetic valve 100, has been implanted. In such instances, the physician may access the coronary vessel through the outflow end 106 of the prosthetic valve by passing through the elongated cells 134. This allows a physician to access the coronary vessel without needing to remove or displace the prosthetic heart valve 100. For example, FIG. 22 illustrates prosthetic valve 100 (with the skirt 120 and valvular structure 122 removed for purposes of illustration) implanted within a patient's native aortic heart valve 800. As shown, the frame 102, once expanded, can retain the native leaflets 802 in an open position against the aortic walls 804. The elongated cells 134 allow a coronary catheter 806 to access to the coronary vessels 808 via the coronary ostia 810. The height of the elongated cells 134 can be selected such that the outflow ends 106 of one or more elongated cells 134 abut the ceiling of the native sinus positioning the prosthetic valve 100 within the native aortic valve 800 and the aortic root such that the coronary vessels 808 remain accessible. Further details of leaflet heights and ratios with prosthetic valve frames can be found, for example, in International Application No. PCT/US2021/025869, incorporated herein by reference in its entirety.

In some instances, it may be necessary to implant a second prosthetic valve within a previously-implanted prosthetic valve in what is known as a valve-in-valve (VIV) procedure. Such a procedure can be used to augment or replace a previously-implanted valve (e.g., if the previously-implanted valve is failing or otherwise compromised). Implantation of the second prosthetic valve or “guest valve” within the first prosthetic valve or “host valve” can be challenging with smaller diameter valves because it can be difficult to properly align and orient the guest valve within the host valve while maintaining access to the coronary ostia. The configuration of prosthetic valve 100 can advantageously retain access to the coronary ostia regardless of the positioning of the guest valve within the host valve.

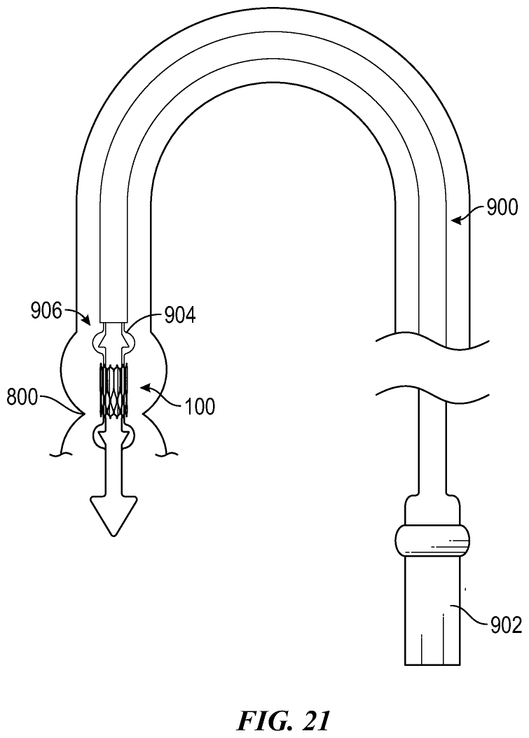

Referring to FIG. 21, a delivery apparatus 900 including a handle 902 can be used to deliver and implant the prosthetic valve 100 in the following exemplary manner. The prosthetic valve 100 can be disposed on a distal end portion 906 of the delivery apparatus 900 in a radially compressed state. The prosthetic valve 100 can be crimped on an inflatable balloon 904 or another type of expansion member that can be used to radially expand the prosthetic valve 100. The distal end portion 906 of the delivery apparatus 900, including prosthetic valve 100, can be advanced through the vasculature to a selected implantation site (e.g., within a previously implanted host valve and/or within a native valve). In the illustrated embodiment, the distal end portion of the delivery apparatus 900 and the prosthetic valve 100 are inserted into a femoral artery and advanced through the femoral artery and the aorta and positioned within the native aortic valve 800 or a host valve previously implanted within the native aortic valve 800. The prosthetic valve 100 can then be deployed at the implantation site, such as by inflating the balloon 904. Further details of delivery apparatuses that can be used to deliver and implant plastically expandable prosthetic valves, such as the prosthetic valve 100 (or any other prosthetic valves disclosed herein) are disclosed in U.S. Pat. Nos. 10,588,744, 10,076,638, and 9,339,384, which are incorporated herein by reference in their entireties.

If the prosthetic valve 100 being implanted is a self-expandable prosthetic valve, the prosthetic valve can be retained in a radially compressed state within a delivery capsule or sheath of the delivery apparatus when inserted into and advanced through the patient's vasculature to the desired implantation site. Once positioned at the desired implantation site, the prosthetic valve can be deployed from the delivery capsule, which allows the prosthetic valve to self-expand to its radially expanded, functional size within the native valve or a previously implanted host valve. Further details of delivery apparatuses that can be used to deliver and implant self-expandable prosthetic valves (including any of the prosthetic valves disclosed herein when the frames are constructed of a self-expandable material such as Nitinol) are disclosed in U.S. Pat. Nos. 9,867,700 and 8,652,202, which are incorporated herein by reference in their entireties.

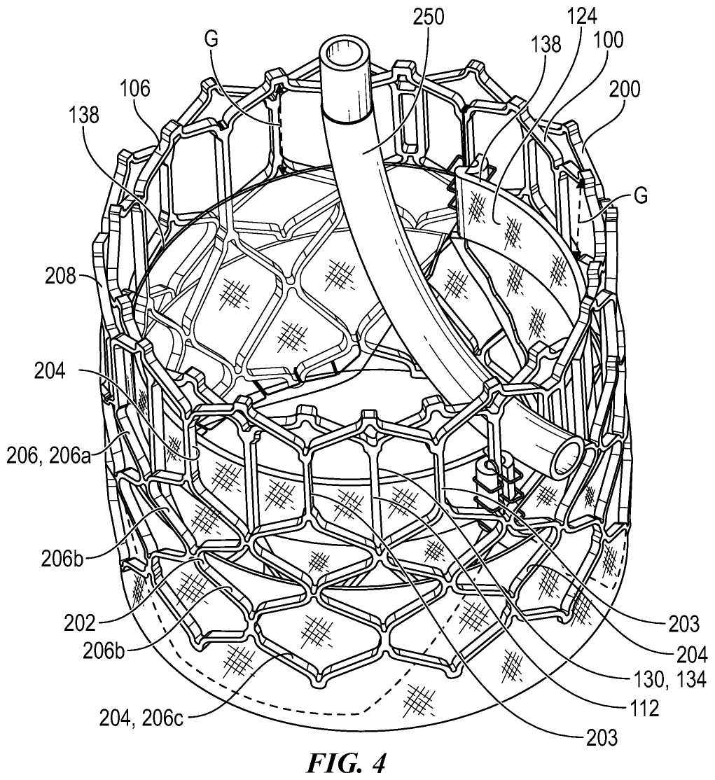

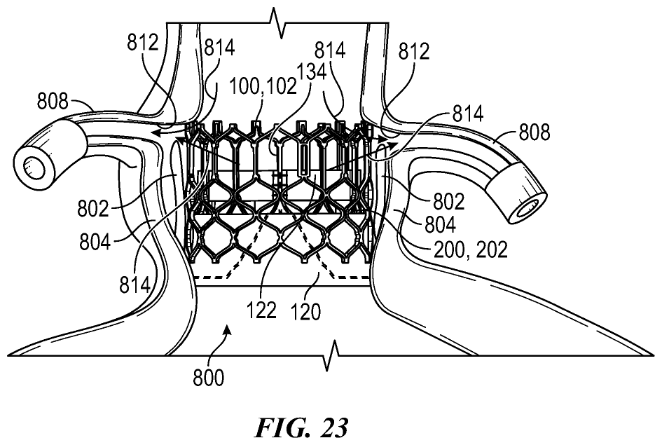

In a particular example, the prosthetic valve 100 can be deployed within a previously implanted host valve 200, as shown in FIG. 23. FIG. 4 illustrates the result of a valve-in-valve procedure using an exemplary balloon-expandable prosthetic valve 200 as the host valve and prosthetic valve 100 as the guest valve. The back-left leaflet 124 (in the orientation shown in FIG. 4) of the guest valve's 100 valvular structure is shown transparently for purposes of illustration. Examples of balloon-expandable prosthetic valves can be found, for example, in U.S. Pat. No. 9,393,110. Host valve 200 can comprise a frame 202 including a plurality of struts 203 forming cells 204 arranged in a plurality of circumferentially extending rows 206. In the illustrated embodiment, the host valve 200 has four rows of cells including an outflow row of cells 206a, two middle rows of cells 206b, and an inflow row of cells 206c. The host valve 200 can further comprise a valvular structure, and inner and/or outer skirts, however, such components are not shown for purposes of illustration

Ideally, the guest valve 100 is implanted in a rotationally aligned position relative to the host valve 200. However, in some instances, the guest valve 100 can be implanted in a rotationally offset position and/or become rotationally offset from the host valve 200 during or after the implantation procedure. As used herein the term “rotationally aligned” means that the struts 112 of the outflow row of cells 130 (the elongated cells 134) of the guest valve 100 are in a rotational position such that they are aligned with the struts 203 of the outflow row of cells 206a of the host valve 200. The term “rotationally offset” means that the struts 112 of the elongated cells 134 are in a rotational position such that they are offset from the struts 203 of the outflow row of cells 206a of the host valve 200 (see e.g., FIG. 4).

FIG. 4 illustrates the guest valve 100 in a “worst-case” rotationally offset position relative to the host valve 200. As used herein, a “worst-case” position means a rotationally offset position in which the struts 112 of the guest valve 100 align with the center of the cells 204 of the outflow row 206a of the host valve 200, or vice versa, resulting in a relatively smaller opening through which a coronary catheter can be inserted. However, the elongated cells 134 of the guest valve 100 have a width W₁ (FIG. 3B) configured such that even when the host valve 200 and guest valve 100 are in a “worst-case” position a coronary catheter 250 (e.g., a 6 Fr coronary catheter) can extend through both the guest valve 100 and the host valve 200, as shown in FIG. 4. The height H₁ (FIG. 3B) of the elongated cells 134 can be selected such that the outflow end 106 of the guest valve frame 100 can abut the ceiling of the native sinus. The gap G between the outflow end 106 of the frame and the outflow edge 138 of the leaflets 124 can serve to allow access to the coronary ostia.

In some embodiments, such as when the host valve 200 comprises a valvular structure aligned with (or substantially aligned with) the outflow end 208 of the frame 202, it may be necessary to cut or remove the valvular structure of the host valve 200 prior to implantation of the guest valve 100. If such action is not taken, the valvular structure can cover the outflow row 206a of cells, obstructing the sinus and potentially harming the patient. However, in the illustrated embodiment, the height H₁ of the elongated cells 134 can further serve as a spacer between the outflow edge of the host valve's 200 valvular structure and the outflow end 106 of the guest valve 100. Accordingly, even if the valvular structure of the host valve 200 is retained by the guest valve 100 in an open configuration against the frame 202, the elongated cells 134 define a space between the ceiling of the native sinus and the outflow edge of the host valvular structure such that the host valvular structure does not occlude the coronary sinus.

For example, FIG. 23 illustrates a guest prosthetic valve 100 (including frame 102, skirt 120, and valvular structure 122) implanted in a valve-in-valve configuration within a host valve 200 in a patient's native aortic heart valve 800. As shown, the previously implanted host valve 200 retains the native leaflets 802 in an open position against the aortic walls 804. The soft components of the host valve 200 (the leaflets and the skirts) are omitted for purposes of illustration. The valve-in-valve configuration can abut the ceiling 812 of the native sinus and serve as a spacer such that the coronary vessels 808 remain accessible. Accordingly, the coronary vessels 808 remain accessible and blood can flow through the frames 102, 202 and into the coronary vessels 808 as shown by arrows 814.

As shown, the width W₁ (FIG. 3B) of each elongated cell 134 is configured to be at least twice as wide as the outer diameter of the selected coronary catheter 250 (e.g., a 6 Fr coronary catheter). Accordingly, the coronary catheter 250 can be disposed through the outflow end row 132a of cells of the guest valve 100 (e.g., the elongated cells 134) and the host valve 200 regardless of the relative rotational orientation between the guest valve 100 and the host valve 200.

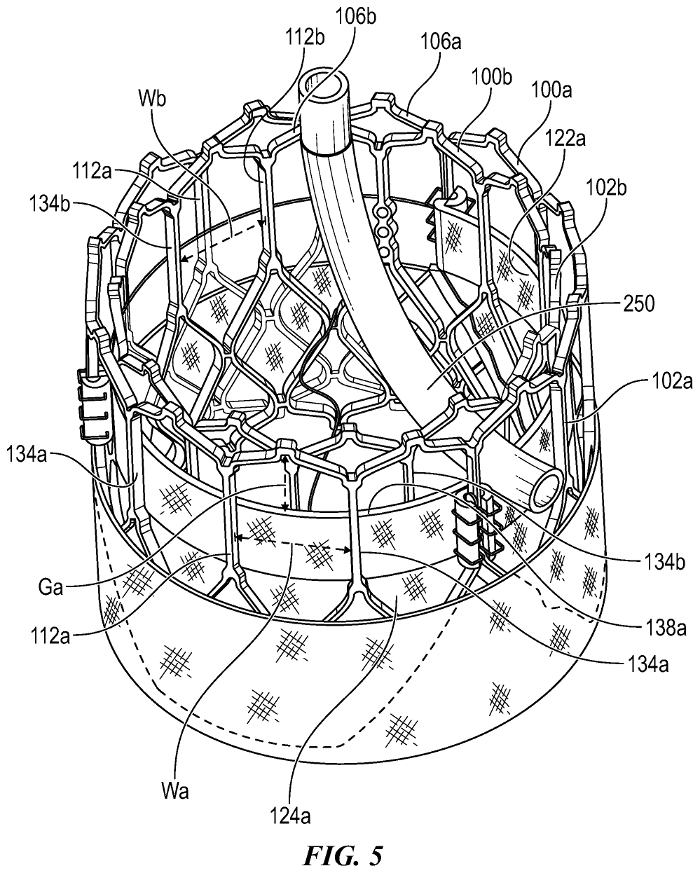

FIG. 5 illustrates the result of a valve-in-valve procedure using a first small diameter valve 100a as the host valve and a second small diameter valve 100b as the guest valve. Guest valve 100b can comprise a valvular structure, an inner skirt, and/or an outer skirt, however such components are not shown for purposes of illustration. Further, the back-left leaflet (in the orientation shown in FIG. 5) of the host valve 100a is not shown for purposes of illustration. FIG. 5 illustrates the guest valve 100b in a “worst-case” rotationally offset position relative to the host valve 100a such that the struts 112b of the guest valve 100b align with the center of the elongated cells 134a of the host valve 100a.

As shown, the elongated cells 134b of the guest valve 100b have a width W_{b} and the elongated cells 134a of the host valve 100a have a width Wₐ such that a coronary catheter 250 (e.g., a 6 Fr coronary catheter) can be inserted through the host and guest elongated cells 134a, 134b, respectively, regardless of the rotational position of the host valve 100a and the guest valve 100b relative to one another. The height of each set of elongated cells 134a, 134b can be selected such that the outflow end 106a, 106b of the host valve frame 100a and the guest valve frame 100b can abut the ceiling of the native sinus when implanted in a patient.

The valvular structure 122a of the host valve 100a can be sized such that the gap Gₐ between the outflow edge 138a of each leaflet 124a and the outflow end 106a of the frame 102a allows the coronary catheter 250 to extend therethrough when the valvular structure 122a is fully open (e.g., as shown in FIG. 5). Such a configuration advantageously allows the guest valve 100b to be implanted within the host valve 100a (thereby maintaining the valvular structure 122a in the fully open configuration) without needing to remove or cut the valvular structure 122a of the host valve 100a.

As shown in FIG. 5, the width Wₐ, W_{b} of each elongated cell 134a, 134b is configured to be at least twice as wide as the outer diameter of the selected coronary catheter 250 (e.g., a 6 Fr coronary catheter). Accordingly, the coronary catheter 250 can be disposed through the elongated cells 134a, 134b of the guest valve 100b and the host valve 100a regardless of the relative rotational orientation between the guest valve 100b and the host valve 100a.

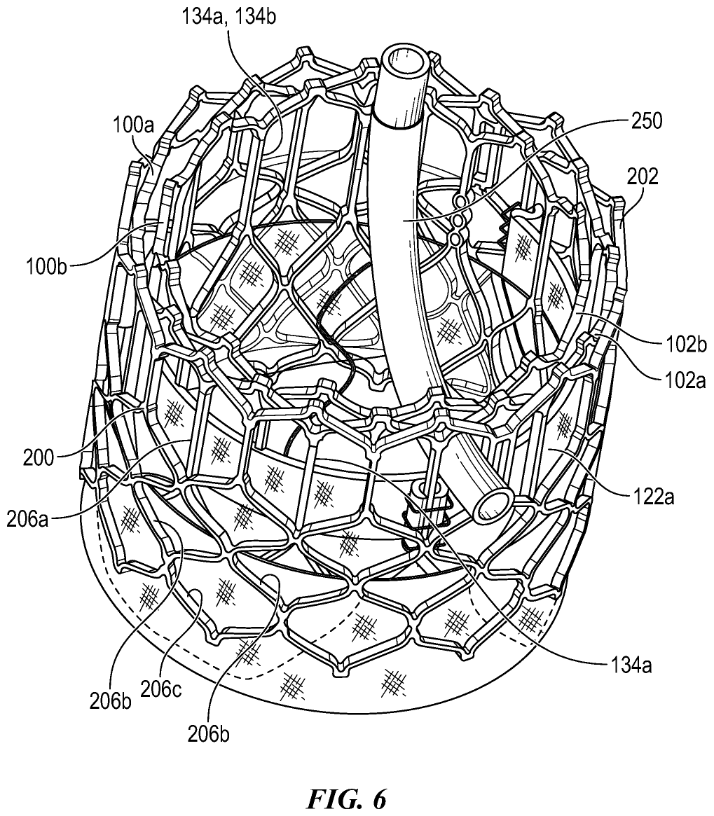

FIG. 6 illustrates result of a VIV procedure using an exemplary balloon-expandable prosthetic valve 200 as the host valve, a first small diameter prosthetic valve 100a as a first guest valve, and a second small diameter prosthetic valve 100b as a second guest valve. The host valve 200 and the second guest valve 100b can each include a valvular structure, an inner skirt, and/or an outer skirt, however these components are omitted for purposes of illustration. Further, the back-left leaflet (in the orientation shown in FIG. 6) of the first guest valve 100a has been omitted for purposes of illustration.

As shown in FIG. 6, regardless of the rotational orientation between the host valve 200, the first guest valve 100a, and the second guest valve 100b, a coronary catheter 250 (e.g., a 6 Fr coronary catheter) can be disposed through the elongated cells 134 of the first and second guest valves 100a, 100b and the outflow end row of cells 206a of the host valve 200. In some embodiments, the host valve 200 can also be configured as a small diameter prosthetic valve.

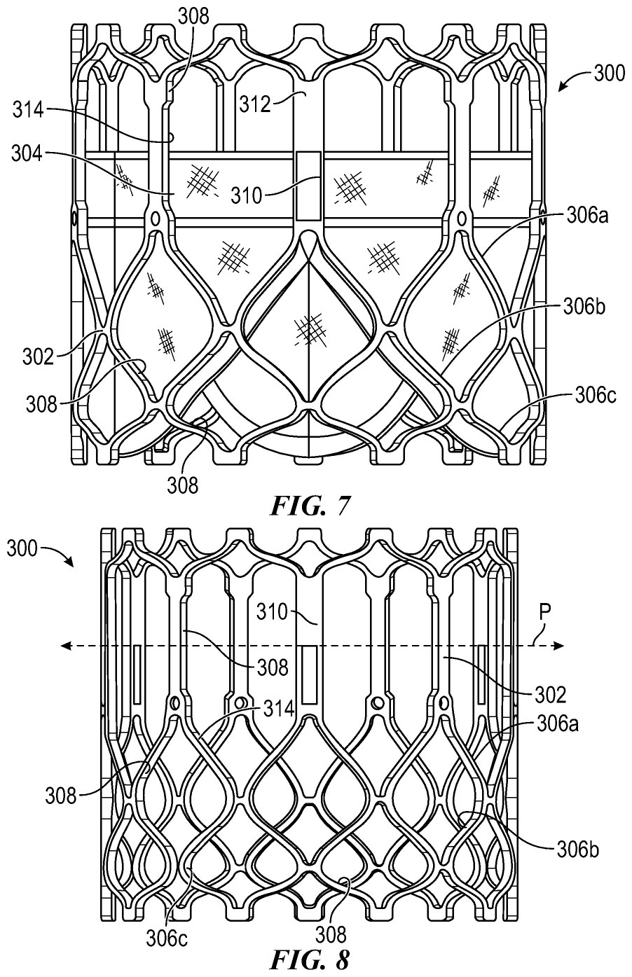

In the illustrated embodiment, the host valve 200 has a 12 cell configuration, and each of the small diameter guest valves 100 can have a 9 cell configuration. “12 cell” and “9 cell” configurations refer to the number of cells in each circumferentially extending row. FIGS. 7-8 illustrate an exemplary prosthetic valve 300 including a frame 302 having a 9 cell configuration. FIG. 7 illustrates the frame 302 of the prosthetic valve 300 coupled to an exemplary valvular structure 304. FIG. 8 illustrates the frame 302 without the valvular structure 304. The prosthetic valve 300 can further comprise inner and/or outer skirts, however, such components are not shown for purposes of illustration. The frame 302 can comprise a plurality of commissure windows 310 and can further comprise three circumferentially-extending rows 306 of cells 308. For example, the frame 302 can comprise an outflow row 306a, a middle row 306b, and an inflow row 306c. Similar to frame 202 described above, the cells 308 outflow row 306a can have a relatively larger open cell area compared to the cells of rows 306b, 306c, and can be referred to as elongated cells 314. The height of the elongated cells 314 in combination with the positioning of the valvular structure 304 within the frame 302 defines a gap between the outflow end of the elongated cells 314 and the outflow edge 316 of the valvular structure 304 configured to accommodate a coronary catheter there-through. As shown in the illustrated embodiment, an outflow edge 312 of the commissure window 310 can be disposed such that it substantially aligns with a plane P (FIG. 8) that is perpendicular to the longitudinal axis of the frame 302 and extends through each of the elongated openings 314.

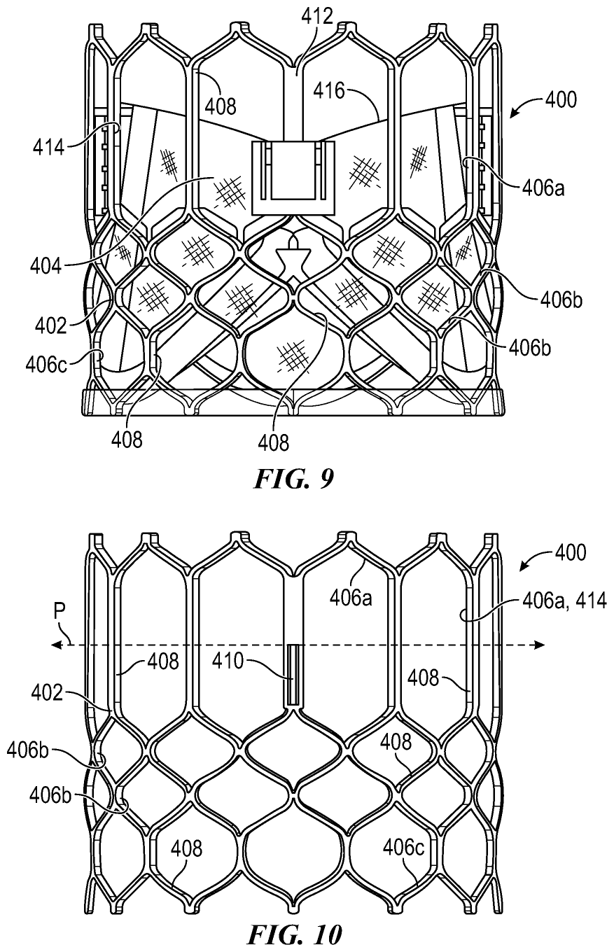

FIGS. 9-10 illustrate an exemplary prosthetic valve 400 having a 12-cell configuration. FIG. 9 illustrates the frame 402 of the prosthetic valve 400 coupled to an exemplary valvular structure 404. FIG. 10 illustrates a portion of the frame 402 without the valvular structure 404. While only one side of the frame 402 is depicted in FIG. 10, it should be appreciated that frame 402 forms an annular structure having an opposite side that is identical (or substantially identical) to the portion shown. The frame 402 can comprise four circumferentially-extending rows 406 of cells 408. For example, the frame 402 can comprise an outflow row 406a, two middle rows 406b, and an inflow row 406c. Similar to frames 202 and 302 described above, the cells 408 outflow row 406a can have a relatively larger open cell area compared to the cells of rows 406b, 406c, and can be referred to as elongated cells 414. The height of the elongated cells 414 in combination with the positioning of the valvular structure 404 within the frame 402 defines a gap between the outflow end of the elongated cells 414 and the outflow edge 416 of the valvular structure 404 configured to accommodate a coronary catheter there-through. As shown in the illustrated embodiment, an outflow edge 412 of the commissure window 410 (FIG. 10) can be disposed such that it substantially aligns with a plane P (FIG. 10) that is perpendicular to the longitudinal axis of the frame 402 and extends through each of the elongated openings 414.

Though the above frame embodiments are described in the context of small-diameter valves, it should be understood that elongated cells and commissure posts such as those described can be used on prosthetic valves having any of various diameters.

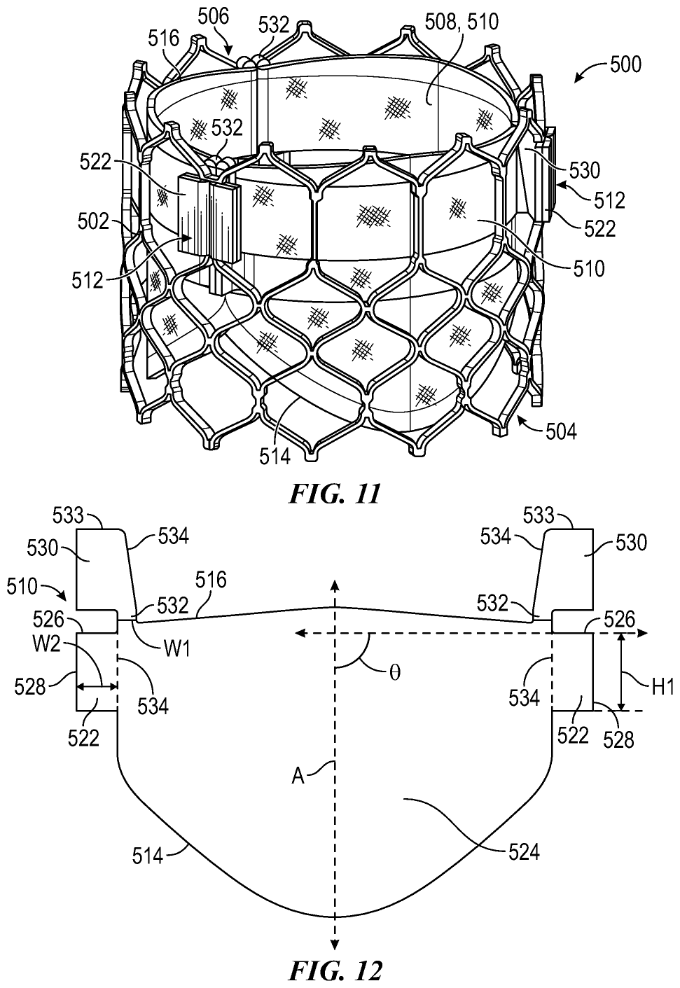

FIGS. 11-14 illustrate another embodiment of a small-diameter prosthetic valve 500. The small diameter prosthetic valve 500 can comprise a frame 502 having an inflow end portion 504 and an outflow end portion 506 and a valvular structure 508 coupled to and supported by the frame 502. The prosthetic valve 500 can further comprise inner and/or outer skirts, however, such components are not shown for purposes of illustration.

The valvular structure 508 is configured to regulate the flow of blood through the prosthetic valve 500 from the inflow end portion 504 to the outflow end portion 506. The valvular structure 508 can include, for example, a leaflet assembly comprising one or more leaflets 510 made of flexible material. The leaflets 510 can be made in whole or in part, from biological material, bio-compatible synthetic materials, or other such materials. Suitable biological material can include, for example, bovine pericardium (or pericardium from other sources). The leaflets 510 can be secured to one another at their adjacent sides to form commissures 512, each which can be secured to a commissure support member, as discussed further below. As shown in FIG. 12, each leaflet can have an inflow edge portion 514 (also referred to as a cusp edge portion) that can be mounted to the frame 502 and an outflow edge portion 516 (also referred to as the free edge portion) that contacts respective outflow edges of the other leaflets during closure of the leaflets (e.g., during diastole).

During typical valve operation, the leaflets 510 transition between a closed state in diastole, with their outflow edges 516 coapting against each other, and an open state (see e.g., FIG. 11) allowing blood to flow through the prosthetic valve 500. The outflow orifice through which the blood can flow determines the pressure gradient across the valve. Known valves can have valvular structures attached to the frame in such a manner that the outflow edges of each leaflet are spaced radially inward of the frame to prevent leaflet abrasion when the leaflets open under the flow of blood. In such valves, the effective outflow orifice (e.g., as determined by the position of the leaflets), also referred to as the geometric orifice area (GOA), can be narrower than the inflow orifice, producing a relatively high pressure gradient across the prosthetic valve. The increased pressure gradient can lead to prosthesis-patient-mismatch (PPM) where the prosthetic valve is essentially undersized for the patient, which has been shown to be associated with worsened hemodynamic function and more cardiac events. Accordingly, and particularly when small diameter valves are used, it is preferable to provide a large outflow orifice during systole to prevent elevated pressure gradients.

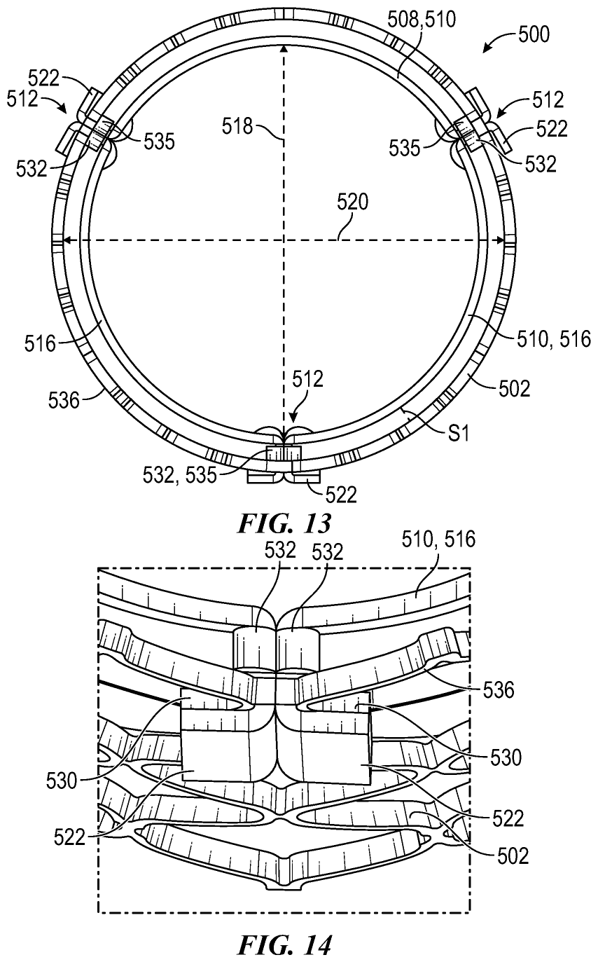

As shown in FIG. 13, the valvular structure 508 of prosthetic valve 500 advantageously defines a relatively large GOA 518 when compared to the size of the outflow orifice 520 defined by the outflow end 506 of the frame. The term “GOA,” as used herein, is defined as the open space through which blood can flow when the valvular structure is in the open configuration. The GOA 518 of the outflow orifice 520 can be sized to provide a selected pressure gradient across the prosthetic valve 500. Such a configuration can be achieved by attaching the leaflets 510 to the frame 502 in such a manner that the radial distance S₁ between the outflow edges 516 of the leaflets 510 and the frame 502 is minimized.

Referring again to FIG. 12, the cusp edge portion 514 terminates at its upper ends at two laterally projecting integral lower tabs 522. The lower tabs 522 can extend from a body 524 of the leaflet 510 such that an upper or outflow edge 526 of each lower tab 522 is positioned at an angle θ relative to a longitudinal axis A of the leaflet 510. The angle θ can be selected such that the radially outer edge 528 of each lower tab 522 corresponds to the draft angle of the frame 502. The “draft angle” of the frame, as used herein, means the degree of taper from the outflow end 506 of the frame 502 to the inflow end 504, which can be a measure of the angle between a longitudinal axis of the frame and a line drawn tangent to the outer surface of the frame 502. For example, in a cylindrical valve, the draft angle is about 0 degrees. In a non-cylindrical, tapered valve (e.g., a frustoconical, V-shaped, or Y-shaped valve), the draft angle can be, for example, between about 2 degrees and about 15 degrees.

In the illustrated embodiment, the frame 502 has a cylindrical shape and the lower tabs 522 are positioned such that the radially outer edge 528 corresponds to (e.g., is substantially parallel to) the draft angle of the frame 502. Accordingly, the lower tabs 522 extend from the body portion 524 such that the angle θ is a 90 degree angle. Such a configuration can advantageously allow for a greater GOA 518 while preventing or mitigating abrasion of the leaflets 510.

In other embodiments wherein the frame has a non-cylindrical shape, the lower tabs 522 can be disposed such that an upper edge 526 of each tab 522 extends at a non-90 degree angle relative to the longitudinal axis of the leaflet (e.g., as shown in FIG. 20). In such embodiments, the angle θ can be less than or greater than 90 degrees. For example, a non-cylindrical valve can have a draft angle between about 2 degrees and about 5 degrees. In such embodiments, the angle θ can be between about 88 degrees and about 85 degrees.

Each lower tab 522 can have a height Hi. The height H₁ can be shorter than the height of a conventional leaflet tab in order to provide a greater GOA during systole. For example, a conventional commissure opening can have a height of about 3.3 mm and conventional leaflets can have a height of 3.7 mm. Accordingly, a conventional leaflet tab must be squeezed in order to fit into a conventional commissure opening, which can form a rigid portion of leaflet that extends radially inward toward the longitudinal axis of the prosthetic valve. In contrast, the height H₁ of lower tabs 522 can be selected to minimize squeezing, and therefore minimize rigid portions formed by the leaflets 510. In some embodiments, the height H₁ of the lower tabs can substantially correspond to the height of the commissure opening. For example, the height of a tab “substantially corresponds” with the commissure opening if the height H₁ of the tab is between 0.1 mm to 0.5 mm greater or less than of the height of the opening. For example, in some particular embodiments, the height H₁ of each lower tab 522 can be 3.4 mm, and the height of the commissure opening can be 3.3 mm.

As shown in FIG. 12, each lower tab 522 can be coupled to an upper tab 530 via a respective neck portion 532. In the illustrated embodiment, each upper tab 530 and neck portion 532 are formed integrally with the leaflet 510. However, in other embodiments, the upper tabs 530 and/or neck portions 532 can be formed separately from the leaflet 510 and coupled to the leaflet 510. In the illustrated embodiment, the upper tab 530 can have a substantially rectangular shape including a radially inner edge portion 534 that tapers from the neck portion 532 to a free edge 533. However, in other embodiments, the upper tab 530 can have any of various shapes. As shown in FIG. 11, when the valvular structure 508 is coupled to the frame 502, each upper tab 530 is folded downward (e.g., toward the inflow end 504 of the frame 502) along the neck portion 532 such that the free edge 533 faces the inflow end 504 of the prosthetic valve 500.

As best seen in FIG. 13, the neck portion 532 can be sized such that when the upper tab 530 is folded downwards (e.g., toward the inflow end 504 of the frame 502), a rigid portion 535 is formed by the folded neck portion 532 that extends radially into the outflow orifice a distance S₁, thereby preventing the leaflets 510 from hitting the frame 502 (preventing or mitigating abrasion and/or other damage to the leaflets) while maximizing the GOA 518 of the outflow orifice 520. Such a configuration advantageously improves the pressure gradient across the valve 500 while minimizing wear along the hinge line 536 (FIG. 12) (e.g., the portion of the leaflet 510 where the lower tab 522 meets the body 524).

In some embodiments such as the illustrated embodiment, the neck portion 532 can have a width W₁ that is less than half the width W₂ of the lower tabs 522 and/or the upper tabs 530. For example, in some particular embodiments, each neck portion 532 can have a width of about 0.70 mm and each lower tab can have a width of about 2.0 mm. In other embodiments, the width W₁ of the neck portion 532 can be half the width W₂ of the lower tabs 522 and/or the upper tabs 530.

Furthermore, the size of the neck portion 532 (and therefore the size of the rigid portion 535 and of the distance S₁) can be varied depending on the specific anatomical needs of the patient. For example, for larger prosthetic valves mounted against dilated anatomical structures, it may be beneficial to reduce the GOA of the outflow orifice. In such cases, the neck portion 532 can be enlarged such that the rigid portion 535 and therefore the space S₁ between the frame 502 and the leaflets 510 extends radially a greater distance into the outflow orifice 520, thereby limiting the flow through the outflow end of the valve 506. In other embodiments, the GOA 518 can also be reduced by, for example, varying the angle of the lower tabs 522 and/or enlarging the height of the lower tabs 522.

Referring to FIG. 14, in some embodiments, the valvular structure 508 can be secured to the frame 502 in the following exemplary manner. Adjacent lower tabs 522 of two adjacent leaflets 510 can be coupled together, and the upper tabs 530 can be folded downward along the neck portions 532 such that the lower tabs 522 are disposed between them. The lower tabs 522 can then be inserted through a commissure window in the frame 520 and folded along the radially outer surface 538 of the frame 502. Each lower tab 522 can be coupled to a respective upper tab 530 along a suture line. In some embodiments, a wedge (not shown) can be disposed between the lower tabs 522 where they fold along the radially outer surface 538 of the frame 502. The wedge can be secured to the lower tabs 522 using one or more sutures.

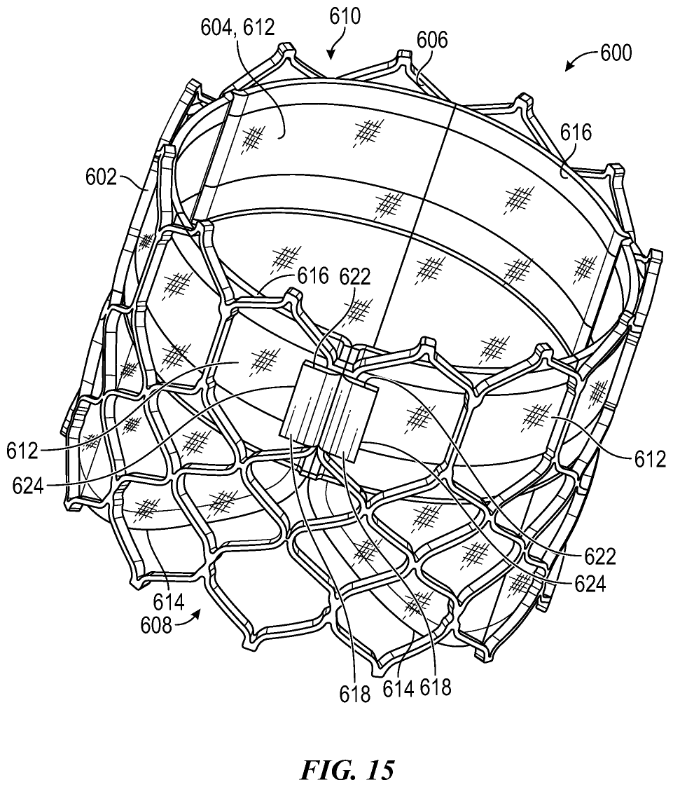

FIGS. 15-17 illustrate another embodiment of a small-diameter prosthetic valve 600 having a frame 602 coupled to a valvular structure 604 configured to provide a selected GOA (e.g., a maximized GOA) at the outflow orifice 606. As shown in FIG. 15, the frame 602 can have an inflow end portion 608 and an outflow end portion 610. The valvular structure 604 can be coupled to and supported by the frame 602. The prosthetic valve 600 can further comprise inner and/or outer skirts, however, such components are not shown for purposes of illustration.

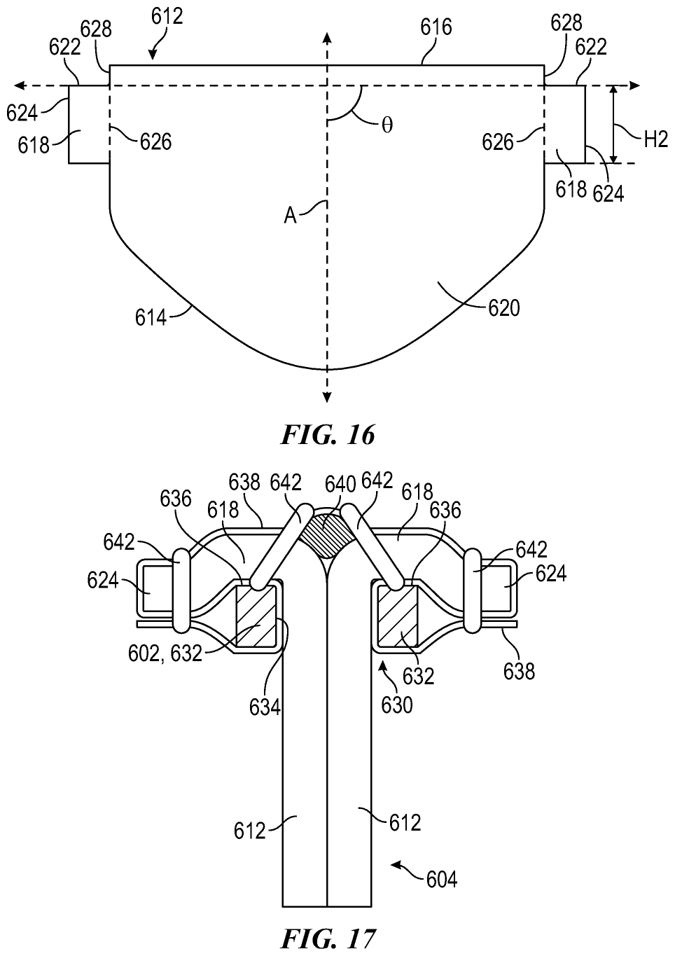

The valvular structure 604 can be similar to the valvular structure 508, described previously, except that the leaflets 612 of valvular structure 604 do not include upper tabs. Referring to FIG. 16, each leaflet 612 can comprise an inflow edge portion 614 (also referred to as a cusp edge portion) that can be mounted to the frame 602 and an outflow edge portion 616 (also referred to as the free edge portion) that contacts respective outflow edges of the other leaflets during closure of the leaflets (e.g., during diastole).

The cusp edge portion 614 of each leaflet 612 terminates at its upper ends at two laterally projecting integral tabs 618. The tabs 618 can extend from a body portion 620 the leaflet 612 such that an upper or outflow edge 622 of each tab 618 is positioned at an angle θ relative to a longitudinal axis A of the leaflet 612. The angle θ can be selected such that the radially outer edge 624 of each tab 618 corresponds to the draft angle of the frame 602. For example, in the illustrated embodiment, the prosthetic valve 600 comprises a cylindrical frame 602 having a draft angle of about 0 degrees. As shown, the tabs 618 extend from the body 620 of the leaflet 612 such that the angle θ is a 90 degree angle relative to a longitudinal axis A of the leaflet 612. In other words, the tabs 618 are positioned such that the radially outer edge 624 corresponds to the draft angle of the frame 602. Such a configuration can result in “streamlining” where the end of the hinge line 626 of each tab 618 is tangent with the commissure folding line, which therefore does not create a rigid portion of the leaflet 618.

Referring still to FIG. 16, each tab 618 can have a height H₂. The height H₂ can be shorter than the height of a conventional leaflet tab in order to provide a larger GOA during systole. A conventional commissure opening can have a height of about 3.3 mm and conventional leaflets can have a height of, for example, about 3.7 mm. Accordingly, a conventional leaflet tab must be squeezed in order to fit into a conventional commissure opening, which can form a rigid portion of leaflet that extends radially inward toward the longitudinal axis of the prosthetic valve. In contrast, the height H₂ of tabs 618 can be selected to minimize squeezing, and therefore minimize rigid portions of the leaflets 612. In some embodiments, the height H₂ of the lower tabs can substantially correspond to the height of the commissure opening. For example, the height of a tab “substantially corresponds” with the commissure opening if the height H₂ of the tab is between 0.1 mm to 0.5 mm greater than or less than of the height of the commissure opening. For example, in some particular embodiments, the height H₂ of each lower tab 522 can be 3.4 mm, and the height of the commissure opening can be 3.3 mm.

Each tab 618 can comprise a step portion 628 disposed between the outflow edge 616 of the leaflet 612 and the upper edge 622 of each tab 618. The step portion 628 is configured to offset the folding area to the junction between the tab's upper edge 622 and the step portion 628, rather than positioning the fold at the upper edge of the leaflet 612. The step portion 628 can further provide a visual indicator to facilitate proper positioning of the tab 618 within the commissure window 630 (FIG. 17) during assembly of the prosthetic valve 600.

In some embodiments, the valvular structure 604 can be secured to the frame 602 in the following exemplary configuration. FIG. 17 illustrates a cross-sectional view of a portion of the frame 602 and valvular structure 604 showing the tabs 618 of adjacent leaflets 612 secured to a commissure window 630. The commissure window 630 can comprise two members 632 defining an opening 634 between them. The tabs 618 can be inserted through the opening 634 and can be folded along the radially outer surface 636 of the frame 602. A flexible connector 638 can extend around each member 632, around the outer edges 624 of each tab 618 and across the radially outer surface of the tabs 618. A wedge 640 can be disposed radially between the flexible connector 638 and the tabs 618 and between the adjacent tabs 618. The various components can then be coupled together using one or more sutures 642.

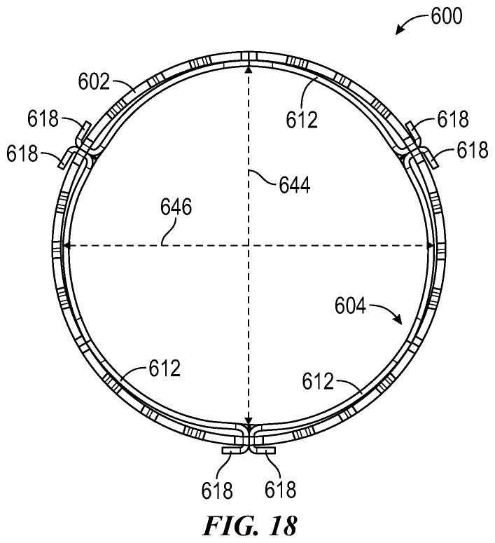

As best seen in FIG. 18, the configuration of the leaflets 612 advantageously allows the valvular structure 604 to be coupled to the frame 602 such that the GOA 644 is nearly the entire area of the outflow orifice 646, thereby maximizing the GOA. In other words, when the valvular structure 604 is in the open configuration, at least a portion of the leaflets 612 abut the frame 602. Such a configuration advantageously improves the pressure gradient across the valve 600. The leaflet parameters, including tab angle and tab height, can be varied to provide a selected GOA 644, for example, to maximize the GOA 644 in a small-diameter prosthetic valve. In some particular embodiments, the configuration of prosthetic valve 600 can result in a GOA of about 216 mm², which can result in a pressure gradient of about 5 mmHg across the prosthetic valve 600.

In some cases, the leaflets 612 can be configured to deliberately reduce the GOA 644, for example, by enlarging the height H of the tabs 618 and/or angling the tabs 618 relative to the draft angle of the frame 602. Reduction of the GOA can be advantageous for, for example, large frames mounted against dilated anatomical structures.

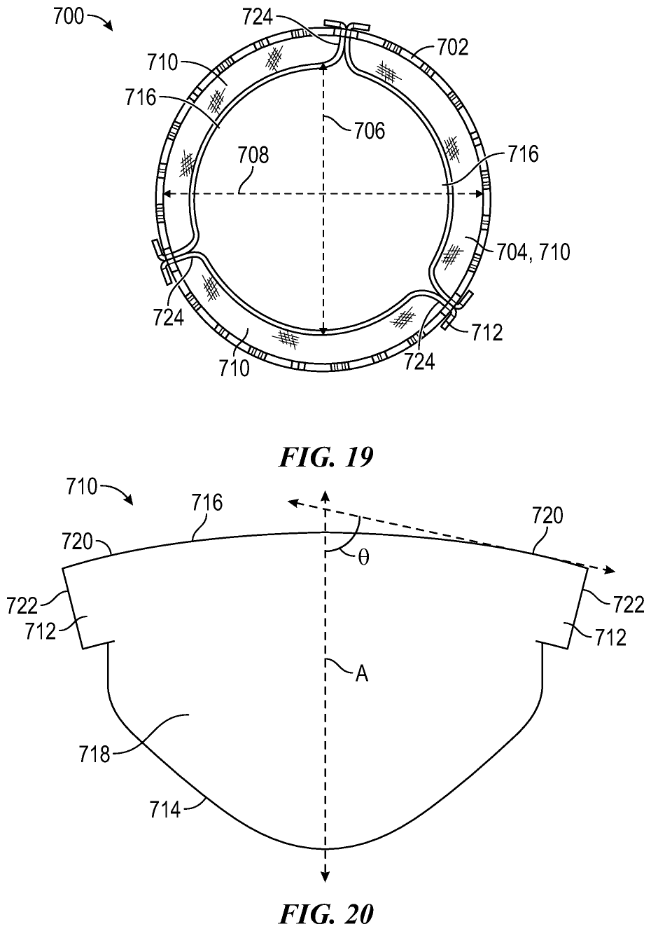

FIGS. 19-20 illustrate another embodiment of a small-diameter prosthetic valve 700 including a valvular structure 704 configured to provide a selected GOA 706 at the outflow orifice 708. As shown in FIG. 19, the prosthetic valve 700 can comprise a frame 702 having an inflow end portion (not shown) and an outflow end portion 710, the valvular structure 704 can be coupled to and supported by the frame 702. The prosthetic valve 700 can further comprise inner and/or outer skirts, however, such components are not shown for purposes of illustration.

The valvular structure 704 can be similar to the valvular structures 508 and 604, described previously, except that the leaflets 710 of the valvular structure 704 have tabs 712 that are angled relative to the draft angle of the frame such that the GOA 706 of the prosthetic valve 700 is reduced when compared to the GOAs of prosthetic valves 500 and 600.

Referring to FIG. 20, each leaflet 710 can comprise an inflow edge portion 714 (also referred to as a cusp edge portion) that can be mounted to the frame 702 and an outflow edge portion 716 (also referred to as the free edge portion) that contacts respective outflow edges of the other leaflets during closure of the leaflets (e.g., during diastole).

The cusp edge portion 714 of each leaflet 710 terminates at its upper ends at two laterally projecting integral tabs 712. The tabs 712 can extend from a body portion 718 the leaflet 710 such that an upper or outflow edge 720 of each tab 712 is positioned at an angle θ relative to a longitudinal axis A of the leaflet 710. In some embodiments, it may be desirable to create a GOA 706 that is narrower than the outflow orifice 708. For example, in embodiments wherein relatively larger frames are mounted against dilated anatomical structures. In such embodiments, the tabs 712 can extend from the body 718 of the leaflet at a non-90 degree angle such that the radially outer edge 722 of each tab 712 does not correspond to the draft angle of the frame 702, thereby forming rigid portions 724 (FIG. 19) where the tabs 712 are coupled to the frame 702. The rigid portions 724 can extend radially inward toward a longitudinal axis of the prosthetic valve 700, as shown in FIG. 19.

In the illustrated embodiment, the outflow edge 720 of each tab 712 is positioned such that the angle θ is less than 90 degrees. In other embodiments, the outflow edge 720 can be positioned such that the angle θ is greater than 90 degrees. As shown in FIG. 19, when coupled to the frame 702, the tabs 712 form rigid portions 724 that extend radially inwardly toward a longitudinal axis of the prosthetic valve 700. The rigid portions 724 space the outflow edge 716 of the leaflets 710 inwardly by a distance S₂, thereby reducing the GOA 706 of the outflow orifice 708. The angle θ can be selected to provide rigid portions 724 of a selected size, thereby selecting a specific GOA for the prosthetic valve 700. The leaflet parameters, including tab angle and tab height, can be varied to provide a selected GOA 706, for example, a reduced GOA.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An implantable prosthetic device, comprising:

a frame movable between a radially compressed configuration and a radially expanded configuration, the frame having an inflow orifice, an outflow orifice, and comprising one or more commissure windows;

a valvular structure comprising a plurality of leaflets, each leaflet comprising a main body having an inflow edge, an outflow edge, and a pair of opposing tabs extending from opposite sides of the main body, each tab being paired with an adjacent tab of an adjacent leaflet to form a commissure tab assembly, each commissure tab assembly being coupled to a respective commissure window; and

wherein each tab extends from the main body at an angle such that a radially outer edge of the tab corresponds to a draft angle of the frame.

Example 2. The prosthetic device of any example herein, particularly example 1, wherein an outflow edge of each tab is spaced apart from the outflow edge of the leaflet by a stepped portion.

Example 3. The prosthetic device of any example herein, particularly any one of examples 1-2, wherein the tabs have a height selected such that the commissure tab assembly fits within a respective commissure window without forming a radially-extending rigid portion.

Example 4. The prosthetic device of any example herein, particularly any one of examples 1-3, wherein each tab has a height that substantially corresponds with a height of the commissure window.

Example 5. The prosthetic device of any example herein, particularly any one of examples 1-4, wherein an outflow edge of the tab is disposed at a 90 degree angle relative to a longitudinal axis of the leaflet.

Example 6. The prosthetic device of any example herein, particularly any one of examples 1-5, wherein each tab is a lower tab and wherein each leaflet further comprises a pair of upper tabs extending from the main body and coupled to the main body via a neck portion, each upper tab comprising a radially inner edge, a radially outer edge, and a free edge.

Example 7. The prosthetic device of any example herein, particularly example 6, wherein each neck portion has a first width and each lower tab has a second width, and wherein the first width is less than half the second width.

Example 8. The prosthetic device of any example herein, particularly any one of examples 6-7, wherein the radially inner edge of each upper tab tapers toward a free edge of the upper tab.

Example 9. The prosthetic device of any example herein, particularly any one of examples 6-8, wherein the upper tabs and neck portions of each leaflet are formed integrally with the main body of the respective leaflet.

Example 10. The prosthetic device of any example herein, particularly any one of examples 6-9, wherein each upper tab is folded along the neck portion toward the inflow edge of the leaflet to form a rigid portion that extends radially inwardly toward a longitudinal axis of the frame.

Example 11. The prosthetic device of any example herein, particularly any one of examples 6-10, wherein a width of the neck portion is selected to provide a selected geometric orifice area (GOA) of the outflow orifice.

Example 12. An implantable prosthetic device, comprising:

a cylindrical frame movable between a radially compressed configuration and a radially expanded configuration;

a valvular structure comprising a plurality of leaflets, each leaflet comprising a main body having an inflow edge, an outflow edge, and a pair of opposing tabs extending from opposite sides of the main body; and

wherein each tab extends from the main body such that an outflow edge of the tab is disposed at a 90-degree angle relative to a longitudinal axis of the leaflet.

Example 13. The prosthetic device of any example herein, particularly example 12, wherein an outflow edge of each tab is spaced apart from the outflow edge of the leaflet by a stepped portion.

Example 14. The prosthetic device of any example herein, particularly any one of examples 12-13, wherein the frame comprises one or more commissure openings, and wherein each tab has a height 0.1 mm greater than a height of the commissure opening.

Example 15. The prosthetic device of any example herein, particularly any one of examples 12-14, wherein the tabs are lower tabs and wherein each leaflet further comprises opposing upper tabs coupled the outflow edge of the leaflet via a neck portion, wherein each lower tab is paired with an adjacent lower tab of an adjacent leaflet to form a commissure, and wherein each upper tab is folded toward an inflow end of the frame such that the neck portion forms a rigid portion extending radially inwardly toward a longitudinal axis of the frame.

Example 16. The prosthetic device of claim 15, wherein each neck portion has a first width and each lower tab has a second width, and wherein the first width is less than half the second width.

Example 17. The prosthetic device of any example herein, particularly example 16, wherein the radially inner edge of each upper tab tapers toward a free edge of the upper tab.

Example 18. The prosthetic device of any example herein, particularly any one of examples 15-17, wherein the upper tabs and neck portions of each leaflet are formed integrally with the main body of the respective leaflet.

Example 19. The prosthetic device of any example herein, particularly any one of examples 15-18, wherein each upper tab is folded along the neck portion toward the inflow edge of the leaflet to form a rigid portion that extends radially inwardly toward a longitudinal axis of the frame.

Example 20. The prosthetic device of any example herein, particularly any one of examples 15-19, wherein a width of the neck portion is selected to provide a selected geometric orifice area (GOA) of the outflow orifice.

Example 21. The prosthetic device of any example herein, particularly any one of examples 15-20, wherein the lower tabs have a height selected such that the lower tab fits within a respective commissure window without forming a radially-extending rigid portion.

Example 22. The prosthetic device of any example herein, particularly any one of examples 15-21, wherein each lower tab has a height that substantially corresponds with a height of the commissure window.

Example 23. The prosthetic device of any example herein, particularly any one of examples 12-22, wherein each tab is paired with an adjacent tab of an adjacent leaflet to form a commissure, the commissure further comprising a flexible connector configured to extend around one or more struts of the frame to couple the commissure to frame.

Example 24. An implantable prosthetic device, comprising:

a cylindrical frame movable between a radially compressed configuration and a radially expanded configuration, the frame comprising an inflow orifice and an outflow orifice; and

a valvular structure comprising a plurality of leaflets, each leaflet comprising

a main body having an inflow edge and an outflow edge,

a pair of opposing lower tabs extending from opposite sides of the main body, and

a pair of opposing upper tabs extending from and coupled to the outflow edge of the leaflet via respective neck portions;

wherein each lower tab extends from the main body such that an outflow edge of the lower tab is disposed at a 90-degree angle relative to a longitudinal axis of the leaflet; and

wherein each lower tab is paired with an adjacent upper tab of an adjacent leaflet to form a plurality of commissures, and wherein each upper tab is folded toward the inflow orifice of the frame such that the neck portion forms a rigid portion extending radially inwardly toward a longitudinal axis of the frame such that the outflow edge of the leaflet defines a selected geometric orifice area (GOA) within the outflow orifice.

Example 25. The prosthetic device of any example herein, particularly example 24, wherein each neck portion has a first width and each lower tab has a second width, and wherein the first width is less than half the second width.

Example 26. The prosthetic device of any example herein, particularly any one of examples 24-25, wherein each upper tab has an angled radially inner edge.

Example 27. The prosthetic device of any example herein, particularly any one of examples 24-26, wherein the upper tabs and neck portions of each leaflet are formed integrally with the main body of the respective leaflet.

Example 28. The prosthetic device of any example herein, particularly any one of examples 24-27, wherein a width of the neck portion is selected to provide a selected geometric orifice area (GOA) of the outflow orifice.

Example 29. The prosthetic device of any example herein, particularly any one of examples 24-28, wherein an outflow edge of each lower tab is spaced apart from the outflow edge of the leaflet by a stepped portion.

Example 30. The prosthetic device of any example herein, particularly any one of examples 24-29, wherein the lower tabs have a height selected such that the lower tab fits within a respective commissure window without forming a radially-extending rigid portion.

Example 31. The prosthetic device of any example herein, particularly any one of examples 24-30, wherein each lower tab has a height that substantially corresponds with a height of the commissure window.

Example 32. The prosthetic device of any example herein, particularly any one of examples 24-31, wherein each neck portion has a first width and each lower tab has a second width, and wherein the first width is less than half the second width.

Example 33. The prosthetic device of any example herein, particularly any one of examples 24-32, wherein a radially inner edge of each upper tab tapers toward a free edge of the upper tab.

Example 34. The prosthetic device of any example herein, particularly any one of examples 24-33, wherein the frame comprises one or more commissure openings, and wherein each lower tab has a height 0.1 mm greater than a height of the commissure opening.

Example 35. The prosthetic device of any example herein, particularly any one of examples 24-34, wherein each lower tab is paired with an adjacent lower tab of an adjacent leaflet to form a commissure, the commissure further comprising a flexible connector configured to extend around one or more struts of the frame to couple the commissure to frame.

Example 36. An implantable prosthetic device, comprising:

a non-cylindrical frame having an inflow orifice and an outflow orifice, the frame movable between a radially compressed configuration and a radially expanded configuration, the frame having a shape in the radially expanded configuration that tapers from a first diameter at the outflow orifice to a second diameter at the inflow orifice, the second diameter being larger than the first diameter;

a valvular structure comprising a plurality of leaflets, each leaflet comprising a main body having an inflow edge, an outflow edge, and a pair of opposing tabs extending from opposite sides of the main body; and

wherein each tab extends from the main body at an angle such that a radially outer edge of the tab corresponds to a draft angle of the frame.

Example 37. The prosthetic device of any example herein, particularly example 36, wherein each tab extends from the main body such that an outflow edge of the tab is disposed at an angle relative to a longitudinal axis of the leaflet, the angle being less than 90 degrees.

Example 38. The prosthetic device of any example herein, particularly example 37, wherein the angle of the tabs is selected to provide a selected geometric orifice area (GOA) of the outflow orifice.

Example 39. The prosthetic device of any example herein, particularly any one of examples 37-38, wherein the angle of the tabs is between about 88 degrees and about 85 degrees.

Example 40. The prosthetic device of any example herein, particularly any one of examples 36-39, wherein each tab is coupled to an adjacent tab of an adjacent leaflet to form a commissure, and wherein each commissure is coupled to the frame such that the tabs form rigid portion that extends radially inwardly toward a longitudinal axis of the frame.

Example 41. The prosthetic device of any example herein, particularly any one of examples 36-40, wherein an outflow edge of each tab is spaced apart from the outflow edge of the leaflet by a stepped portion.

Example 42. The prosthetic device of any example herein, particularly any one of examples 36-41, wherein each tab has a height that substantially corresponds with a height of a commissure window defined in the frame.

Example 43. The prosthetic device of any example herein, particularly example 36, wherein an outflow edge of the tab is disposed at a 90 degree angle relative to a longitudinal axis of the leaflet.

Example 44. The prosthetic device of any example herein, particularly any one of examples 36-43, wherein each tab is a lower tab and wherein each leaflet further comprises a pair of upper tabs extending from the main body and coupled to the main body via a neck portion, each upper tab comprising a radially inner edge, a radially outer edge, and a free edge.

Example 45. The prosthetic device of any example herein, particularly example 44, wherein each neck portion has a first width and each lower tab has a second width, and wherein the first width is less than half the second width.

Example 46. The prosthetic device of any example herein, particularly any one of examples 44-45, wherein the radially inner edge of each upper tab tapers toward a free edge of the upper tab.

Example 47. The prosthetic device of any example herein, particularly any one of examples 44-46, wherein the upper tabs and neck portions of each leaflet are formed integrally with the main body of the respective leaflet.

Example 48. The prosthetic device of any example herein, particularly any one of examples 44-47, wherein each upper tab is folded along the neck portion toward the inflow edge of the leaflet to form a rigid portion that extends radially inwardly toward a longitudinal axis of the frame.

Example 49. The prosthetic device of any example herein, particularly any one of examples 44-48, wherein a width of the neck portion is selected to provide a selected geometric orifice area (GOA) of the outflow orifice.

Example 50. The prosthetic device of any example herein, particularly any one of examples 44-49, wherein the frame comprises one or more commissure openings, and wherein each lower tab has a height 0.1 mm greater than a height of the commissure opening.

Example 51. An implantable prosthetic device, comprising:

an annular frame that is movable between a radially compressed configuration and a radially expanded configuration, the frame comprising first, second, and third circumferentially-extending rows of cells, the first row of cells disposed adjacent an outflow end of the frame;

a valvular structure comprising a plurality of leaflets, each leaflet having a main body including an inflow edge and an outflow edge, and a pair of opposing tabs extending from opposite sides of the main body, each tab being paired with an adjacent tab of an adjacent leaflet and secured to the frame to form a commissure assembly;

wherein the valvular structure is secured to the frame such that a gap is defined between the outflow edges of the leaflets and the outflow end of the frame; and
wherein each cell of the first row of cells is configured to be at least twice as wide as a selected coronary catheter.

Example 52. The implantable device of any example herein, particularly example 51, wherein one or more struts of the first row of cells are commissure posts comprising a plurality of apertures.

Example 53. The implantable device of any example herein, particularly example 52, wherein each commissure post comprises three apertures.

Example 54. The implantable device of any example herein, particularly any one of examples 51-53, wherein the inflow edges of the leaflets are coupled to an inflow end of the frame by one or more sutures extending through the leaflets and around struts of the frame that define the inflow end of the frame.

Example 55. The implantable device of any example herein, particularly any one of examples 51-54, wherein the prosthetic device is devoid of any fabric material inside of the frame.

Example 56. The implantable device of any example herein, particularly any one of examples 51-55, wherein the commissure assemblies are devoid of any fabric material.

Example 57. The implantable device of any example herein, particularly any one of examples 51-56, wherein the cells in the first row of cells each have a height greater than the cells in the second and third rows of cells.

Example 58. The implantable device of any example herein, particularly any one of examples 51-57, wherein the cells in the first row of cells have a height selected to allow access to the coronary vessel through the gap when the implantable device is implanted within a native annulus.

Example 59. A method, comprising:

inserting a distal end of a delivery apparatus into the vasculature of a patient, the delivery apparatus releasably coupled to a guest prosthetic valve movable between a radially compressed and a radially expanded configuration, the prosthetic valve including a frame comprising first, second, and third circumferentially-extending rows of cells, the first row of cells disposed adjacent an outflow end of the frame and configured to be at least twice as wide as a selected coronary catheter, and a valvular structure disposed within the frame and coupled to the frame such that a gap is defined between the outflow edges of the valvular structure and an outflow end of the frame;

advancing the guest prosthetic valve to a selected implantation site comprising a previously implanted host prosthetic valve, the host prosthetic valve comprising a host frame and a host valvular structure disposed within the host frame;

positioning the guest prosthetic valve within the host prosthetic valve; and radially expanding the guest prosthetic valve within the previously implanted host prosthetic valve.

Example 60. The method of any example herein, particularly example 59, further comprising inserting the selected coronary catheter through the gap of the guest prosthetic valve and through the frame of the host prosthetic valve.

Example 61. The method of any example herein, particularly any one of examples 59-60, further comprising cutting the host valvular structure prior to radially expanding the guest prosthetic valve.

Example 62. The method of any example herein, particularly any one of examples 59-61, wherein the host frame comprises first, second, and third circumferentially-extending rows of cells, the first row of cells disposed adjacent an outflow end of the host frame and configured to be at least twice as wide as a selected coronary catheter, and the host valvular structure is coupled to the host frame such that a gap is defined between the outflow edges of the host valvular structure and an outflow end of the host frame.

Example 63. The method of any example herein, particularly example 62, further comprising inserting the selected coronary catheter through the gap of the guest prosthetic valve and the gap of the host prosthetic valve.

Example 64. The method of any example herein, particularly any one of examples 59-63, wherein the guest prosthetic valve is rotationally offset relative to the host prosthetic valve.

Example 65. A method of assembling a prosthetic heart valve, comprising:

forming a valvular structure from a plurality of leaflets, each leaflet comprising an inflow edge, an outflow edge, and two opposing tabs, wherein the valvular structure is formed by coupling adjacent tabs of adjacent leaflets to one another to form respective commissures;

positioning the valvular structure within a radially expandable and compressible frame, the frame comprising first, second, and third circumferentially-extending rows of cells, the first row of cells disposed adjacent an outflow end of the frame and configured to be at least twice as wide as a selected coronary catheter; and

coupling the valvular structure to the frame such that a gap is defined between the outflow edges of each leaflet and an outflow edge of the frame when the valvular structure is in an open configuration.

Example 66. An assembly, comprising:

a first implantable prosthetic device and a second implantable prosthetic device, each implantable prosthetic device comprising:
an annular frame that is movable between a radially compressed configuration and a radially expanded configuration, the frame comprising first, second, and third circumferentially-extending rows of cells, the first row of cells disposed adjacent an outflow end of the frame,
a valvular structure comprising a plurality of leaflets, each leaflet having a main body including an inflow edge and an outflow edge, and a pair of opposing tabs extending from opposite sides of the main body, each tab being paired with an adjacent tab of an adjacent leaflet and secured to the frame to form a commissure assembly, the valvular structure secured to the frame such that a gap is defined between the outflow edges of the leaflets and the outflow end of the frame; and

wherein each cell of the first row of cells is configured to be at least twice as wide as a selected coronary catheter; and

wherein the first implantable prosthetic device is disposed withing the annular frame of the second implantable prosthetic device.

Example 67. The assembly of any example herein, particularly example 66, wherein the first prosthetic device is rotationally offset relative to the second prosthetic device.

Example 68. The assembly of any example herein, particularly any one of examples 66-67, wherein the selected coronary catheter can extend through the gap in the first prosthetic device and the gap in the second prosthetic device.

In view of the many possible embodiments to which the principles of the disclosure may be applied, it should be recognized that the illustrated embodiments are only preferred examples and should not be taken as limiting the scope. Rather, the scope is defined by the following claims. We therefore claim all that comes within the scope and spirit of these claims.

We claim:

1. An implantable prosthetic device, comprising:
a frame movable between a radially compressed configuration and a radially expanded configuration, the frame having an inflow orifice, an outflow orifice, and comprising one or more commissure windows;
a valvular structure comprising a plurality of leaflets, each leaflet comprising a main body having an inflow edge, an outflow edge, and a pair of opposing tabs extending from opposite sides of the main body, each tab being paired with an adjacent tab of an adjacent leaflet to form a commissure tab assembly, each commissure tab assembly being coupled to a respective commissure window; and
wherein each tab extends from the main body at an angle such that a radially outer edge of the tab corresponds to a draft angle of the frame.

2. The prosthetic device of claim 1, wherein an outflow edge of each tab is spaced apart from the outflow edge of the leaflet by a stepped portion.

3. The prosthetic device of claim 1, wherein the tabs have a height selected such that the commissure tab assembly fits within a respective commissure window without forming a radially-extending rigid portion.

4. The prosthetic device of claim 1, wherein each tab has a height that substantially corresponds with a height of the commissure window.

5. The prosthetic device of claim 1, wherein an outflow edge of the tab is disposed at a 90 degree angle relative to a longitudinal axis of the leaflet.

6. The prosthetic device of claim 1, wherein each tab is a lower tab and wherein each leaflet further comprises a pair of upper tabs extending from the main body and coupled to the main body via a neck portion, each upper tab comprising a radially inner edge, a radially outer edge, and a free edge.

7. The prosthetic device of claim 6, wherein each neck portion has a first width and each lower tab has a second width, and wherein the first width is less than half the second width.

8. The prosthetic device of claim 6, wherein the radially inner edge of each upper tab tapers toward a free edge of the upper tab.

9. The prosthetic device of claim 6, wherein the upper tabs and neck portions of each leaflet are formed integrally with the main body of the respective leaflet.

10. The prosthetic device of claim 6, wherein each upper tab is folded along the neck portion toward the inflow edge of the leaflet to form a rigid portion that extends radially inwardly toward a longitudinal axis of the frame.

11. The prosthetic device of claim 6, wherein a width of the neck portion is selected to provide a selected geometric orifice area (GOA) of the outflow orifice.

12. An implantable prosthetic device, comprising:
a cylindrical frame movable between a radially compressed configuration and a radially expanded configuration;
a valvular structure comprising a plurality of leaflets, each leaflet comprising a main body having an inflow edge, an outflow edge, and a pair of opposing tabs extending from opposite sides of the main body; and
wherein each tab extends from the main body such that an outflow edge of the tab is disposed at a 90-degree angle relative to a longitudinal axis of the leaflet.

13. The prosthetic device of claim 12, wherein an outflow edge of each tab is spaced apart from the outflow edge of the leaflet by a stepped portion.

14. The prosthetic device of claim 12, wherein the frame comprises one or more commissure openings, and wherein each tab has a height 0.1 mm greater than a height of the commissure opening.

15. The prosthetic device of claim 12, wherein the tabs are lower tabs and wherein each leaflet further comprises opposing upper tabs coupled the outflow edge of the leaflet via a neck portion, wherein each lower tab is paired with an adjacent lower tab of an adjacent leaflet to form a commissure, and wherein each upper tab is folded toward an inflow end of the frame such that the neck portion forms a rigid portion extending radially inwardly toward a longitudinal axis of the frame.

16. The prosthetic device of claim 15, wherein each neck portion has a first width and each lower tab has a second width, and wherein the first width is less than half the second width.

17. The prosthetic device of claim 16, wherein the radially inner edge of each upper tab tapers toward a free edge of the upper tab.

18. The prosthetic device of claim 15, wherein the upper tabs and neck portions of each leaflet are formed integrally with the main body of the respective leaflet.

19. The prosthetic device of claim 15, wherein each upper tab is folded along the neck portion toward the inflow edge of the leaflet to form a rigid portion that extends radially inwardly toward a longitudinal axis of the frame.

20. The prosthetic device of claim 15, wherein a width of the neck portion is selected to provide a selected geometric orifice area (GOA) of the outflow orifice.
